(19) United States
(12) Patent Application Publication
(10) Pub. No.: US 2024/0285860 A1
(43) Pub. Date: Aug. 29, 2024

(54) MEDICATION DELIVERY SYSTEMS, APPARATUSES AND METHODS

(71) Applicant: SADLEIR LABORATORIES PTY LTD, Cottesloe (AU)

(72) Inventors: Paul Harold Martin SADLEIR, Cottesloe (AU); John Willoughby SADLEIR, Cottesloe (AU)

(73) Assignee: SADLEIR LABORATORIES PTY LTD, Cottesloe (AU)

(21) Appl. No.: 18/570,586

(22) PCT Filed: Jun. 15, 2022
(86) PCT No.: PCT/AU2022/050593

§ 371 Date: Dec. 14, 2023

(30) Foreign Application Priority Data
Jun. 15, 2021 (AU) 2021901792
Jun. 15, 2021 (AU) 2021901794

(51) Int. Cl. A61M 5/28; A61M 5/14; A61M 5/145; A61M 5/168; A61M 5/19; A61M 5/31

(52) U.S. Cl. CPC A61M 5/284; A61M 5/1409; A61M 5/14526; A61M 5/16827; A61M 5/19; A61M 2005/3128

(57) ABSTRACT

Examples of the present application disclose a medication delivery apparatus comprising a first plunger, a second plunger and a container configured to receive the second plunger and at least a portion of the first plunger. The container and the second plunger define a dilution chamber that is configured to receive a diluent. The container defines a dilution chamber opening. The first plunger, the container and the second plunger define an active agent chamber that is configured to receive a pharmaceutical preparation. The second plunger comprises a one-way valve configured to control a flow of pharmaceutical preparation from the active agent chamber to the dilution chamber. The one-way valve is configured to move from a closed position to an open position upon application of a force exceeding a valve threshold force to an inlet side of the valve. The valve threshold force is less than a sum of a break loose force of the second plunger and a break loose force of the first plunger.

### TECHNICAL FIELD

Described embodiments relate to systems, apparatuses and methods for delivering mixed fluids. In particular, described embodiments replate to systems, apparatuses and methods for administering pharmaceutical preparations.

### BACKGROUND

Administering a pharmaceutical preparations (such as intravenous drugs) to patients can involve a number of risks. The risks are particularly relevant to patients that have a drug hypersensitivity reaction to a particular intravenous drug. Drug hypersensitivity reactions to particular intravenous drugs are typically difficult, if not impossible to predict. In particular, the specific dose of a drug that may induce a drug hypersensitivity reaction in a particular patient is difficult to predict prior to administration of the drug.

In order to reduce the risk of any patient suffering a life-threatening reaction to a drug, one method of administering a particular intravenous drug is to give the patient a specific dose (referred to as a test dose) that would cause a submaximal adverse response. The test dose is delivered prior to the delivery of a therapeutic dose of the drug.

Upon detection of any submaximal or minor adverse reaction in response to the test dose, the administration of the intravenous drug may be immediately aborted to minimise the risk of a more serious adverse reaction developing, or the ultimate death of the patient.

That said, the practice of administering a test dose is not routine nor recommended. This is particularly true because:
The test dose that will typically elicit a submaximal reaction is typically of the order of 0.01% to 0.1% of the therapeutic dose to be given to the patient and the preparation of a test dose of this amount is time-consuming and difficult.
Test doses that will elicit a detectable submaximal reaction vary between patients, potentially varying between 0.01% and 100% of the therapeutic dose. For example, a detectable submaximal reaction may occur for a particular patient at any one of 0.01%, 1%, 10% or 100% of the therapeutic dose, and this varies between patients.

These reasons, among others, make it difficult or even impossible for a clinician to choose an appropriate test dose with which to conduct a trial to confirm whether an adverse reaction will occur during administration of the therapeutic dose. In particular, administering a test dose that is relatively small may not elicit an adverse reaction in the patient; however, administering a relatively large dose (above a specific threshold that is particular to each patient) may cause an adverse reaction that is life-threatening. This reaction may lead to death of the patient. Thus, administering the test dose may lead to a life-threatening condition that the provision of the test dose had the intention of mitigating.

Due the difficulties in determining what specific percentage of the therapeutic dose should be administered to the patient as a test dose for that particular patient, the current practice is to administer intravenous drugs via constant infusion (either a brief ‘push’ or a constant infusion over a fixed time period). This has similar risks to those mentioned above. Administering the therapeutic dose of a drug without confirming whether the patient is hypersensitive or allergic to that particular drug may result in administering a lethal drug dose to the patient, or cause a serious adverse reaction.

Furthermore, currently any test dose that may be administered to a patient is necessarily done prior to, and separate from, infusion of the therapeutic dose that a particular patient requires. Preparation of separate test doses requires preparation of a multitude of pharmaceutical preparations for each test dose and also for the therapeutic dose. This process is cumbersome and therefore, typically, test doses are not provided to patients. Instead the therapeutic dose is provided to the patient without having tested the reaction of the patient to the drug. This increases the risk that particular patients (that have a drug hypersensitivity reaction to a particular drug) may suffer life threatening conditions while being administered this particular drug. This is particularly true because the current methods for administering the full therapeutic dose (a ‘push’ or a constant infusion over a fixed time period) provide relatively large doses at the start of the infusion process compared to that typically required to cause a serious adverse reaction. This does not allow enough time for the clinician to detect that the patient being infused the pharmaceutical preparation is having an adverse (i.e. negative) reaction to the drug.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

A first aspect of the present disclosure provides medication delivery apparatus comprising: a first plunger; a second plunger; and a container configured to receive the second plunger and at least a portion of the first plunger; wherein: the container and the second plunger define a dilution chamber that is configured to receive a diluent; the container defines a dilution chamber opening; the first plunger, the container and the second plunger define an active agent chamber that is configured to receive a pharmaceutical preparation; and the second plunger comprises a one-way valve configured to allow flow of pharmaceutical preparation from the active agent chamber to the dilution chamber and inhibit flow of fluid from the dilution chamber to the active agent chamber.

A second aspect of the present disclosure provides a medication delivery apparatus comprising: a first plunger; a second plunger; and a container configured to receive the second plunger and at least a portion of the first plunger; wherein: the container and the second plunger define a dilution chamber that is configured to receive a diluent; the container defines a dilution chamber opening; the first plunger, the container and the second plunger define an active agent chamber that is configured to receive a pharmaceutical preparation; the second plunger comprises a one-way valve configured to control a flow of pharmaceutical preparation from the active agent chamber to the dilution chamber; the one-way valve is configured to move from a closed position to an open position upon application of a force exceeding a valve threshold force to an inlet side of the valve; and the valve threshold force is less than a sum of a break loose force of the second plunger and a break loose force of the first plunger; wherein the medication delivery apparatus is operable to force pharmaceutical preparation from the active agent chamber through the one-way valve into the dilution chamber to mix with the diluent in the dilution chamber and force the mixed diluent and pharmaceutical preparation out of the dilution chamber through the dilution chamber opening. Mixing of the pharmaceutical preparation with the diluent in the dilution chamber may occur at the same time as diluted pharmaceutical preparation is expelled out of the dilution chamber through the dilution chamber opening.

A third aspect of the present disclosure provides a medication delivery apparatus comprising: a first plunger; a second plunger; and a container configured to receive the second plunger and at least a portion of the first plunger; wherein: the first plunger is configured to seal with the container to provide a first seal; the second plunger is configured to seal with the container to provide a second seal; the container and the second plunger define a dilution chamber that is configured to receive a diluent; the container defines a dilution chamber opening; the first plunger, the container and the second plunger define an active agent chamber that is configured to receive a pharmaceutical preparation; the second plunger comprises a one-way valve configured to control a flow of pharmaceutical preparation from the active agent chamber to the dilution chamber; and a break loose force of the second plunger is greater than a break loose force of the first plunger.

The first, second or third aspects of the present disclosure may have any of the following further features.

The container may define an active agent chamber opening, and an active agent chamber port that comprises an active agent chamber port opening. The active agent chamber port may be positioned on a side of the container between active chamber opening and the dilution chamber opening. For instance, between an initial positon of the first plunger and initial positon of the second plunger.

The valve threshold force may be less than the break loose force of the second plunger. The first plunger may have a concave surface facing the second plunger. The second plunger may have a convex surface facing the first plunger and a convex surface facing the dilution chamber opening.

The first plunger and second plunger may be shaped such that when the first plunger is moved into contact with the second plunger there is an air gap between the first plunger and the second plunger. The air gap may act as a bubble trap. The second plunger may partially conform to the shape of the first plunger so as to minimise wastage of pharmaceutical preparation, while also allowing space for any air bubbles to be trapped between the first plunger and the second plunger when the first plunger is moved into contact with the second plunger.

In some embodiments a distal end of the container defines the dilution chamber opening, the second plunger is movable towards the distal end of the container up to an end position at which the second plunger cannot be moved further towards the distal end of the container and wherein the second plunger is shaped such that in the end position there is a gap between at least part of the second plunger and the distal end of the container so as to prevent a suction force attaching the second plunger to the distal end of the container. The second plunger may have a different shape or profile to a distal end of the container so as to prevent suction of the second plunger to the distal end of the container.

The second plunger may partially conform to the distal end of the container so as to minimise wastage of pharmaceutical preparation and/or diluent when the second plunger is moved into contact with the distal end of the container so as to expel the contents of the dilution chamber. When the second plunger is moved into contact with the distal end of the container, as the second plunger partially conforms to the distal end of the container there is still a gap between at least part of the second plunger and the distal end of the container so as to prevent a suction force adhering the second plunger to the distal end of the container.

In some embodiments, a side of the second plunger facing the distal end of the container includes a first part which abuts against distal end of the container in the end position and a second part which does not abut against the distal end of the container in the end position.

The one-way valve may be a duck-bill valve.

In some embodiments, the one-way valve is contained inside a main body of the second plunger, the main body includes at least one outlet opening and the main body includes at least one internal channel leading from an outlet of the one-way valve to the at least one outlet opening.

In some embodiments, the main body of the second plunger comprises at least two outlet openings.

In some embodiments, the at least two outlet openings are configured to generate a first jet of pharmaceutical preparation directed toward a first corner of the dilution chamber and a second jet of pharmaceutical preparation directed toward a second corner of the dilution chamber when pharmaceutical preparation is forced from the active agent chamber through the one-way valve.

In some embodiments, the apparatus is configured such that the first and second jets of pharmaceutical preparation rebound from an internal surface of the dilution chamber thereby promoting retrograde mixing of the pharmaceutical preparation with diluent in the dilution chamber.

In some embodiments, the one-way valve is duck-bill valve having a slit which is substantially perpendicular to a line joining a first opening and a second opening of the at least two outlet openings.

In some embodiments, the main body of the second plunger comprises three outlet openings.

In some embodiments, the middle opening of the three outlet openings is configured to generate a third jet of pharmaceutical preparation directed toward the dilution chamber opening.

In some embodiments, the second plunger has a length along a longitudinal axis of the container which is at least 9 mm.

In some embodiments, the first plunger comprises a plunger lumen, extending between a first plunger lumen opening and a second plunger lumen opening, for delivering pharmaceutical preparation through the plunger lumen and into the active agent chamber.

In some embodiments, there is further provide a plunger lock to fix a position of the first plunger relative to the container so that the first plunger may not move further into the container; optionally the plunger lock may include a first groove for receiving a projection (e.g. flange) of the first plunger and a second groove for receiving a projection (e.g. flange) of the container.

In some embodiments, the container has a marking indicating a starting position at which the second plunger is to be positioned within the container at a start of an infusion.

A fourth aspect of the present disclosure provides a medication delivery apparatus of any of the above claims in combination with a conduit apparatus, the conduit apparatus comprising: a conduit apparatus housing including: a first housing port; and a second housing port; and a conduit connecting the first housing port and the second housing port; wherein: the first housing port comprises a first connector configured for connection to the dilution chamber outlet opening; and the second housing port comprises a second connector configured for connection to tubing that is to connect to a patient.

A fifth aspect of the present disclosure provides a method of preparing the medication delivery apparatus of the above aspects, the method comprising: a) filling the dilution chamber with a diluent; and b) filling the active agent chamber with pharmaceutical preparation.

In some embodiments, the dilution chamber is filled with diluent prior to the active agent chamber being filled with pharmaceutical preparation.

In some embodiments, the dilution chamber is filled via the dilution chamber opening and the active agent chamber is filled with an active agent port on a side of the container.

In some embodiments, the active agent chamber is filled with active agent prior to the dilution chamber being filled with diluent.

In some embodiments, the active agent chamber is filled with active agent via a plunger lumen of the first plunger.

In on example, prior to filling the dilution chamber, the second plunger is positioned at a starting position which defines an initial volume of the dilution chamber which the dilution chamber is to have at a start of the infusion. The second plunger may be provided to the clinician at an initial position which is the same as the starting position or further away from a distal end of the container than the starting position.

A sixth aspect of the present disclosure provides a method of preparing the medication delivery apparatus comprising an active agent chamber, a dilution chamber, a one-way valve connecting the active agent chamber and the dilution chamber and a dilution chamber opening, the method, comprising: c) connecting the medication delivery apparatus to an infusion driver; d) attaching extension tubing of a known predetermined volume to the dilution chamber opening of the medication delivery apparatus; and e) priming the medication delivery apparatus by causing pharmaceutical preparation to pass from the active agent chamber through the one-way valve into the dilution chamber, mix with diluent in the dilution chamber and then exit through the dilution chamber opening into the extension tubing, such that the extension tubing of known predetermined volume is filled with diluted pharmaceutical preparation which is to form a first part of the infusion and wherein a concentration profile of the diluted pharmaceutical preparation in the extension tubing is in accordance with a desired dose profile for a first part of the infusion.

In some embodiments, after priming the medication delivery apparatus, attaching to the extension tubing to a patient and using the infusion driver to control the medication delivery apparatus to dilute the pharmaceutical preparation by mixing the pharmaceutical preparation with the diluent in the dilution chamber and deliver the diluted pharmaceutical preparation to the patient in accordance with a predetermined dose profile.

A seventh aspect of the present disclosure provides a medication delivery system comprising: the medication delivery apparatus of any one of the above claims; and an infusion device; wherein the infusion device comprises: at least one infusion device processor; and infusion device memory storing program instructions accessible by the at least one infusion device processor, and configured to cause the at least one infusion device processor to control the medication delivery apparatus to deliver the pharmaceutical preparation to a patient in accordance with a predetermined dose profile.

In some embodiments, the infusion device is configured to actuate the infusion device actuator to displace the first plunger such that the pharmaceutical preparation is output by the medication delivery apparatus in accordance with the predetermined dose profile; or the infusion device is configured to apply an infusion pressure at the dilution chamber outlet, thereby causing displacement of the first plunger such that the pharmaceutical preparation is output by the medication delivery apparatus.

In some embodiments the system further comprises an extension tubing of a known predetermined volume to the medication delivery apparatus and the processor is configured to perform a priming process prior to a start of the infusion, wherein the priming process comprises: priming the medication delivery apparatus by causing pharmaceutical preparation to pass from the active agent chamber through the one-way valve into the dilution chamber, mix with diluent in the dilution chamber and then exit through the dilution chamber opening into the extension tubing, such that the extension tubing of known predetermined volume is filled with diluted pharmaceutical preparation which is to form a first part of the infusion and wherein a concentration profile of the diluted pharmaceutical preparation in the extension tubing is in accordance with a desired dose profile for a first part of the infusion.

In some embodiments, there is provided a medication delivery apparatus. The medication delivery apparatus comprises: a first plunger; a second plunger; and a container configured to receive the second plunger and at least a portion of the first plunger. The container and the second plunger define a dilution chamber that is configured to receive a diluent. The container defines a dilution chamber opening. The first plunger, the container and the second plunger define an active agent chamber that is configured to receive a pharmaceutical preparation. The second plunger comprises a valve configured to control a flow of pharmaceutical preparation from the active agent chamber to the dilution chamber. The first plunger comprises a plunger lumen that extends between a first plunger lumen opening and a second plunger lumen opening.

In some embodiments, the active agent chamber is configured to receive the pharmaceutical preparation through the plunger lumen.

In some embodiments, the first plunger comprises a first Luer-lock connector that defines the first plunger lumen opening.

In some embodiments, the first plunger lumen opening is a plunger lumen inlet.

In some embodiments, the second plunger lumen opening is a plunger lumen outlet.

In some embodiments, the first plunger comprises a one-way valve configured to control a flow of pharmaceutical preparation from the plunger lumen to the active agent chamber.

In some embodiments, the medication delivery apparatus further comprises a first plunger cap configured to connect to the first plunger to cap the first plunger lumen opening.

In some embodiments, the first plunger cap comprises a second Luer-lock connector configured to connect with the first Luer-lock connector of the first plunger.

In some embodiments, the first plunger comprises an air lumen that extends between a first air lumen opening and a second air lumen opening.

In some embodiments, the first plunger and the second plunger are each configured to be displaced with respect to a longitudinal axis of the container.

In some embodiments, the second plunger is disposed between the first plunger and the dilution chamber opening.

In some embodiments, the container defines an inner container surface; and the first plunger comprises a first plunger sealing surface that is configured to seal with the inner container surface to inhibit fluid flow between the inner container surface and the first plunger sealing surface.

In some embodiments, the first plunger comprises a first plunger O-ring, the first plunger O-ring comprising the first plunger sealing surface.

In some embodiments, the container defines an inner container surface; and the second plunger comprises a second plunger sealing surface that is configured to seal with the inner container surface to inhibit fluid flow between the inner container surface and the second plunger sealing surface.

In some embodiments, the second plunger comprises a second plunger O-ring, the second plunger O-ring comprising the second plunger sealing surface.

In some embodiments, the valve comprises an inlet side and an outlet side.

In some embodiments, the valve is configured to move from a closed position to an open position upon application of pressure to the inlet side.

In some embodiments, the valve is configured to move from the open position to the closed position upon removal of the pressure applied to the inlet side.

In some embodiments, the valve is biased toward the closed position.

In some embodiments, the valve comprises a plurality of flaps that are configured to separate upon application of pressure to the inlet side.

In some embodiments, the medication delivery apparatus further comprises a conduit configured to be fluidly connected to the dilution chamber opening, the conduit being of a predetermined volume.

In some embodiments, the medication delivery apparatus further comprises a dilution chamber port cap configured to connect to the container to cap the dilution chamber opening.

In some embodiments, the first plunger is configured to seal with the container to provide a first seal.

In some embodiments, the second plunger is configured to seal with the container to provide a second seal.

In some embodiments, a break loose force of the second plunger is greater than a break loose force of the first plunger.

In some embodiments, there is provided a medication delivery apparatus. The medication delivery apparatus comprises: a first plunger; a second plunger; and a container configured to receive the second plunger and at least a portion of the first plunger. The container and the second plunger define a dilution chamber that is configured to receive a diluent. The container defines a dilution chamber opening. The first plunger, the container and the second plunger define an active agent chamber that is configured to receive a pharmaceutical preparation. The second plunger comprises a valve configured to control a flow of pharmaceutical preparation from the active agent chamber to the dilution chamber.

In some embodiments, there is provided a medication delivery apparatus. The medication delivery apparatus comprises: a first plunger; a second plunger; and a container configured to receive the second plunger and at least a portion of the first plunger. The container and the second plunger define a dilution chamber that is configured to receive a diluent. The container defines a dilution chamber opening. The first plunger, the container and the second plunger define an active agent chamber that is configured to receive a pharmaceutical preparation. The second plunger comprises a valve configured to control a flow of pharmaceutical preparation from the active agent chamber to the dilution chamber. The valve is configured to move from a closed position to an open position upon application of a force exceeding a valve threshold force to an inlet side of the valve. The valve threshold force is less than a sum of a break loose force of the second plunger and a break loose force of the first plunger.

In some embodiments, the valve threshold force is less than the break loose force of the second plunger.

In some embodiments, an open valve force of the valve is less than the break loose force of the second plunger.

In some embodiments, the valve is configured to move from the open position to the closed position upon removal of the force applied to the inlet side of the valve.

In some embodiments, there is provided a medication delivery apparatus. The medication delivery apparatus comprises: a first plunger; a second plunger; and a container configured to receive the second plunger and at least a portion of the first plunger. The first plunger is configured to seal with the container to provide a first seal. The second plunger is configured to seal with the container to provide a second seal. The container and the second plunger define a dilution chamber that is configured to receive a diluent. The container defines a dilution chamber opening. The first plunger, the container and the second plunger define an active agent chamber that is configured to receive a pharmaceutical preparation. The second plunger comprises a valve configured to control a flow of pharmaceutical preparation from the active agent chamber to the dilution chamber. A break loose force of the second plunger is greater than a break loose force of the first plunger.

In some embodiments, the container defines an active agent chamber opening.

In some embodiments, the container defines an active agent chamber port that comprises an active agent chamber port opening.

In some embodiments, the first plunger comprises a first number of O-rings.

In some embodiments, the second plunger comprises a second number of O-rings.

In some embodiments, the second number is greater than the first number.

In some embodiments, one or more of the first number of O-rings comprises a first O-ring of a first diameter and a first O-ring groove of a first groove width.

In some embodiments, one or more of the second number of O-rings comprises a second O-ring of a second diameter and a second O-ring groove of a second groove width.

In some embodiments, the second diameter is greater than the first diameter.

In some embodiments, the second plunger comprises a valve arrangement that is configured to control the flow of pharmaceutical preparation from the active agent chamber to the dilution chamber, the valve arrangement comprising the valve.

In some embodiments, the second plunger comprises a second valve.

In some embodiments, the valve and the second valve are configured to control the flow of pharmaceutical preparation from the active agent chamber to the dilution chamber.

In some embodiments, there is provided a medication delivery apparatus. The medication delivery apparatus comprises: a first plunger; a second plunger; a container configured to receive the second plunger and at least a portion of the first plunger; and at least one resistance element configured to resist displacement of the second plunger when the first plunger is displaced. The container and the second plunger define a dilution chamber that is configured to receive a diluent. The container defines a dilution chamber opening. The first plunger, the container and the second plunger define an active agent chamber that is configured to receive a pharmaceutical preparation. The second plunger comprises a valve configured to control a flow of pharmaceutical preparation from the active agent chamber to the dilution chamber.

In some embodiments, the container defines an active agent chamber opening.

In some embodiments, the at least one resistance element comprises a second plunger O-ring.

In some embodiments, the second plunger comprises the second plunger O-ring.

In some embodiments, the first plunger comprises a first number of first plunger O-rings.

In some embodiments, one or more of the first number of first plunger O-rings comprises a first plunger O-ring of a first diameter and a first plunger O-ring groove of a first groove width.

In some embodiments, one or more of the second number of second plunger O-rings comprises a second plunger O-ring of a second diameter and a second plunger O-ring groove of a second groove width.

In some embodiments, the second diameter is greater than the first diameter.

In some embodiments, the second groove width is less than the first groove width.

In some embodiments, the second plunger comprises a valve arrangement that is configured to control the flow of pharmaceutical preparation from the active agent chamber to the dilution chamber, the valve arrangement comprising the valve.

In some embodiments, the second plunger comprises a second valve.

In some embodiments, the valve and the second valve are configured to control the flow of pharmaceutical preparation from the active agent chamber to the dilution chamber.

In some embodiments, the valve is configured to move from a closed position to an open position upon application of a force exceeding a valve threshold force to an inlet side of the valve.

In some embodiments, the valve threshold force is less than a sum of a break loose force of the second plunger and a break loose force of the first plunger.

In some embodiments, a break loose force of the second plunger is greater than a break loose force of the first plunger.

In some embodiments, there is provided a conduit apparatus. The conduit apparatus comprises: a housing comprising: a first housing port; and a second housing port; and a conduit connecting the first housing port and the second housing port. In some embodiments, the first housing port comprises a first connector; and the second housing port comprises a second connector.

In some embodiments, the conduit is coiled within the housing.

In some embodiments, the conduit is of a predetermined volume.

In some embodiments, the housing further comprises a collar that extends away from the second connector in a direction that is parallel to a longitudinal direction of the conduit apparatus.

In some embodiments, the collar is configured to engage with the container.

In some embodiments, the first connector is a third Luer-lock connector that is configured to connect to a dilution chamber port that defines the dilution chamber opening.

In some embodiments, the second connector is a fourth Luer-lock connector.

In some embodiments, there is provided a medication delivery system. The medication delivery system comprises: any one of the medication delivery apparatus embodiments described above; and any one of the conduit apparatus embodiments described above.

In some embodiments, there is provided a medication delivery system. The medication delivery system comprises: any one of the medication delivery apparatuses described above; and an infusion device. The infusion device comprises: at least one infusion device processor; and infusion device memory. The infusion device memory stores program instructions accessible by the at least one infusion device processor, and configured to cause the at least one infusion device processor to: actuate an infusion device actuator to displace the first plunger such that the pharmaceutical preparation is output by the medication delivery apparatus.

In some embodiments, there is provided a medication delivery system. The medication delivery system comprises: any one of the medication delivery apparatus embodiments described above; and an infusion device. The infusion device comprises: at least one infusion device processor; and infusion device memory storing program instructions accessible by the at least one infusion device processor, and configured to cause the at least one infusion device processor to: control the infusion device to apply an infusion pressure at the dilution chamber outlet, thereby causing displacement of the first plunger such that the pharmaceutical preparation is output by the medication delivery apparatus.

### BRIEF DESCRIPTION OF DRAWINGS

Embodiments of the present disclosure will now be described by way of non-limiting example only, with reference to the accompanying drawings, in which:

FIG. 1A is a perspective view of a medication delivery system, according to some embodiments;

FIG. 1B is a block diagram of the medication delivery system illustrated in FIG. 1, according to some embodiments;

FIG. 2 is a side view of a cross section of a medication delivery apparatus, according to some embodiments;

FIG. 3 is a perspective view of the medication delivery apparatus of FIG. 2, where a portion of a container of the medication delivery apparatus is transparent;

FIG. 4 is a perspective view of a medication delivery system, according to some embodiments;

FIGS. 5A to 5E illustrate a method of operation of the medication delivery system, according to some embodiments;

FIG. 6 illustrates a method of operation of the medication delivery apparatus of FIGS. 1 to 5E, according to some embodiments;

FIG. 7 illustrates another method of operation of the medication delivery apparatus of FIGS. 1 to 5E, according to some embodiments;

FIG. 8 illustrates a method for preparing the medication delivery apparatus of FIGS. 1 to 5E, according to some embodiments;

FIG. 9 illustrates another method for preparing the medication delivery apparatus of FIGS. 1 to 5E, according to some embodiments;

FIG. 10 illustrates a medication delivery apparatus having a first plunger with a concave surface facing the second plunger a second plunger having a convex surface facing the first plunger and convex surface facing the distal end of the container, according to some embodiments;

FIG. 11 illustrates a cap for the primary plunger and supporting structure for the cap, according to some embodiments;

FIG. 12 illustrates a medication delivery apparatus comprising a plunger lumen, according to some embodiments;

FIG. 13A is a schematic diagram of a cross section of the medication delivery apparatus of FIG. 12, according to some embodiments;

FIG. 13B is a schematic diagram of a cross section of the medication delivery apparatus of FIG. 12 when filled with a diluent and a pharmaceutical preparation, according to some embodiments;

FIG. 14 illustrates a method of preparing the medication delivery apparatus of FIGS. 12 to 13B, according to some embodiments;

FIG. 15 illustrates an embodiment of the medication delivery apparatus comprising an alternative first plunger and an alternative second plunger, according to some embodiments;

FIG. 16 illustrates a method of preparing the medication delivery apparatus of FIG. 15, according to some embodiments;

FIG. 17 illustrates a method of preparing the medication delivery apparatus, according to some embodiments;

FIG. 18 illustrates another method of preparing the medication delivery apparatus, according to some embodiments;

FIGS. 19A and 19B illustrates a side view of a medication delivery apparatus with the plungers in different positions according to some embodiments;

FIG. 20A illustrates a side view of a medication delivery apparatus that comprises an O-ring, in a first state, according to some embodiments;

FIG. 20B illustrates a side view of another medication delivery apparatus that comprises an O-ring, in the first state, according to some embodiments;

FIG. 20C illustrates a cross-section of an O-ring, according to some embodiments;

FIG. 21 illustrates a side view of a medication delivery apparatus in a first state, according to some embodiments;

FIG. 22 illustrates a side view of a medication delivery apparatus that comprises a projection, in a first state, according to some embodiments;

FIG. 23 illustrates a side view of a partial cross-section of a medication delivery apparatus, according to some embodiments;

FIG. 24 illustrates a side view of a partial cross-section of a medication delivery apparatus, according to some embodiments;

FIG. 25A illustrates a second plunger, according to some embodiments;

FIG. 25B illustrates another second plunger, according to some embodiments;

FIG. 25C illustrates a plunger, according to some embodiments;

FIG. 25D illustrates a plunger, according to some embodiments;

FIG. 26 illustrates a medication delivery apparatus with a first plunger and a second plunger shaped to provide an air trap, according to some embodiments;

FIG. 27 illustrates a second plunger having a single outlet, according to some embodiments;

FIG. 28 illustrates a second plunger having two outlets, according to some embodiments;

FIG. 29A illustrates a second plunger having two outlets, according to some embodiments;

FIG. 29B is a plan view of a second plunger having two outlets and a horizontal duckbill valve, according to some embodiments;

FIG. 29C is a plan view of a second plunger having two outlets and a vertical duckbill valve according to some embodiments;

FIGS. 30A to 30F illustrate jets of pharmaceutical preparation ejected through two outlets of the second plunger into the dilution chamber and mixing of pharmaceutical preparation with diluent, according to some embodiments;

FIG. 31 is a perspective view of a second plunger having three outlets, according to some embodiments;

FIG. 32 is a cross sectional view of a second plunger having three outlets, according to some embodiments;

FIGS. 33A to 33F illustrate jets of pharmaceutical preparation ejected through three outlets of the second plunger into the dilution chamber and mixing of pharmaceutical preparation with diluent, according to some embodiments;

FIG. 34 illustrates an internal structure of a second plunger having a plurality of channels and outlets, according to some embodiments;

FIG. 35 illustrates a medication delivery system, according to some embodiments;

FIG. 36 illustrates a pump having a primary inlet and a secondary inlet which may be connected to a conduit apparatus or medication delivery apparatus according to some embodiments;

FIG. 37 illustrates a perspective view of a conduit apparatus and a portion of a medication delivery apparatus, according to some embodiments; and

FIG. 38 illustrates a perspective view of the conduit apparatus and the medication delivery apparatus of FIG. 37, according to some embodiments.

### DESCRIPTION OF EMBODIMENTS

The present disclosure relates to systems for administering pharmaceutical preparations. In particular, a number of embodiments of a medication delivery apparatus for administering pharmaceutical preparations are disclosed.

It will be understood that the term “active agent” as used in the description, may correspond to, or also be referred to as an “active ingredient” or a “drug”. That is, throughout this disclosure, the terms “active ingredient”, “active agent” and “drug” have been used to describe the active agent that is to be administered to a patient. In some embodiments, a pharmaceutical preparation can be delivered to a patient. The pharmaceutical preparation may comprise the active agent. The pharmaceutical preparation may also comprise one or more other components. For example, the pharmaceutical preparation may comprise a solvent. That is, in some embodiments, the pharmaceutical preparation may comprise the active agent and a solvent. In some embodiments, the pharmaceutical preparation may comprise a diluent. That is, in some embodiments, the pharmaceutical preparation may comprise the active agent, the solvent and/or the diluent. The pharmaceutical preparation may comprise the active agent at a particular concentration. This may be referred to as an active agent concentration. The pharmaceutical preparation may be a solution. It will be understood that in some embodiments, the term “drug” as used in the description may correspond to the active agent of the “pharmaceutical preparation”.

### Medication Delivery System

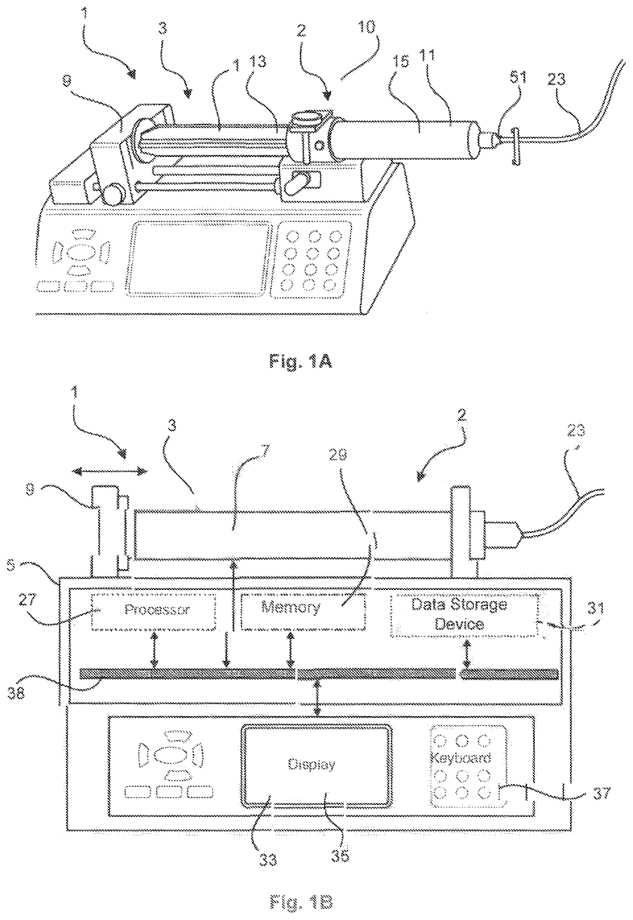

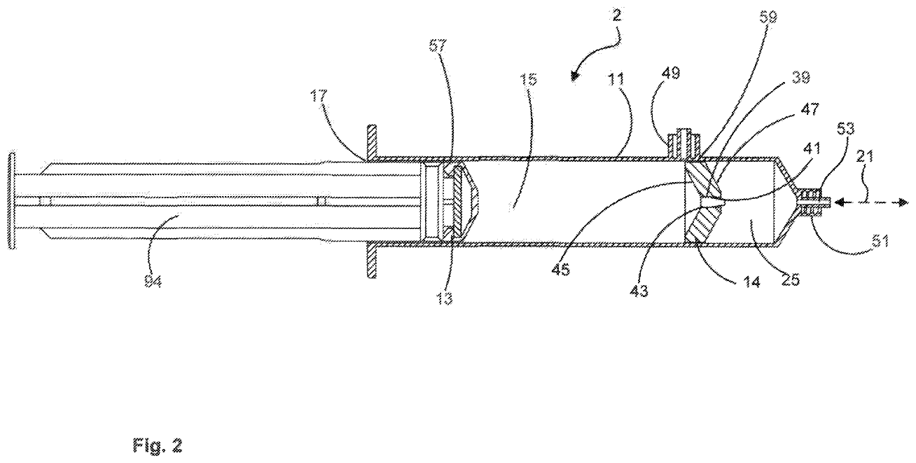

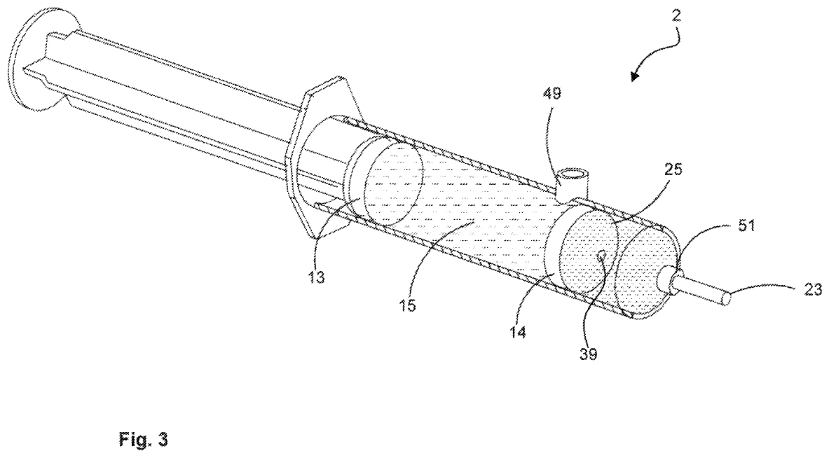

FIGS. 1A, 1B, 4 and 5A to 5E illustrate a system 1, according to some embodiments. In some embodiments, the system 1 is a fluid delivery system 1. In some embodiments, the system 1 is a medication delivery system 1. The medication delivery system 1 comprises a fluid delivery apparatus 2. The fluid delivery apparatus 2 is configured to mix fluids and output a mixed fluid. In some embodiments, the fluid delivery apparatus 2 is in the form of a medication delivery apparatus 2. FIGS. 2, 3, 6 and 7 illustrate the medication delivery apparatus 2, according to some embodiments.

The medication delivery system 1 is configured to provide a pharmaceutical preparation to a patient. The medication delivery system 1 is configured to provide the pharmaceutical preparation in accordance with a target flow rate that approximates a flow rate delivery function. In some embodiments, the medication delivery system 1 is configured to provide the pharmaceutical preparation in accordance with a target flow rate as described in International Patent Application No. PCT/AU2020/051363, the content of which is incorporated herein by reference in its entirety. In some embodiments, the medication delivery system 1 is configured to provide the pharmaceutical preparation in accordance with a target flow rate as described in Australian Provisional Patent Application No. 2021901792, the content of which is incorporated herein by reference in its entirety. The target flow rate may vary over time.

As described in PCT/AU2020/051363 and Australian Provisional Patent Application No. 2021901792, an infusion device may be configured to control a medication delivery apparatus to intravenously deliver a pharmaceutical preparation to a patient, the infusion device comprising a processor and a memory storing instructions executable by the processor to cause the medication delivery apparatus to deliver the pharmaceutical preparation to the patient according to a predetermined dose profile, wherein the predetermined dose profile is designed to deliver a therapeutic dose of the pharmaceutical preparation to the patient over a predetermined infusion time in a manner which facilitates safe detection of an adverse reaction of the patient to the pharmaceutical preparation, or desensitization the patient to the pharmaceutical preparation, during a first stage of administering the therapeutic dose.

In some examples, the predetermined dose profile is such that the dose rate varies over the predetermined infusion time. In some examples, the cumulative dose delivered to the patient increases exponentially, or increases at a rate that increases over time, for at least a portion of the predetermined infusion time. In some examples, the dose profile is such that there is a first time period between the cumulative dose reaching 0.01% and 0.1% and a second time period between the cumulative dose reaching 0.1% and 1% of the therapeutic dose; and the first period of time and the second period of time are selected from the group comprising: at least 6 minutes, at least 5 minutes, at least 4 minutes, at least 3 minutes, between 2 minutes and 10 minutes, and at least the latent period of adverse reaction.

The processor of the infusion device may control the medication delivery apparatus to deliver the pharmaceutical preparation according to the predetermined profile by controlling an infusion device. For example the infusion device may drive a pump or be controlled to drive a plunger of a medication delivery apparatus such that the pharmaceutical preparation is delivered according to the predetermined dose profile. For instance, the processor may divide the predetermined infusion time into a number of infusion steps and determine a target flow rate or a target output volume for each infusion step such that the predetermined dose profile is realized when the actuator is controlled according to the target flow rate or target output volume for each infusion step. The target flow rates or target output volumes for the infusion steps for a predetermined dose profile may be determined by referring to a lookup table stored in the memory or calculated in real time. In any case the cumulative dose of drug delivered to the patient starts at a very low level and increases over the course of the infusion; in some cases the dose rate increases as the infusion progresses. The infusion may, for example, last from 20 to 180 minutes. The dose rate may be relatively low and only increase slowly during a first part of the infusion, e.g. during the first 10 minutes of the infusion.

It can be difficult for many infusion drivers to accurately deliver low infusion rates. Therefore in some examples the medication delivery apparatus may comprise an active agent chamber which expels a pharmaceutical preparation into a separate dilution chamber containing a diluent and diluted pharmaceutical preparation may then flow on to the patient, e.g. through a conduit such as extension tubing. As in this way the pharmaceutical preparation may be heavily diluted at the start of the infusion, higher infusion rates can be used while still delivering a low dose rate. The concentration of pharmaceutical preparation in the dilution chamber and delivered to the patient may vary and may increase over at least part of the infusion. One convenient implementation is a medication delivery apparatus in the form of a syringe having a pair of plungers defining an active agent chamber and a dilution chamber, as will be described in more detail below.

The medication delivery system 1 comprises an infusion device 3 (such as syringe driver, peristaltic pump, volumetric pump or similar drug infusion pump). The infusion device 3 may comprise or be in the form of an infusion driver 3. The infusion device 3 may comprise or be in the form of a vacuum infusion device 3. Where the infusion device 3 comprises or is in the form of the vacuum infusion device 3, the infusion device 3 may apply an infusion pressure (i.e. the vacuum pressure 61) at a dilution chamber opening 53, thereby causing displacement of the first plunger 13 such that the pharmaceutical preparation is output by the medication delivery apparatus 2. The pharmaceutical preparation may be output at a target flow rate.

The infusion device 3 comprises a control unit for controlling the flow rate at which the infusion device 3 delivers the pharmaceutical preparation from the medication delivery apparatus 2 to the patient. The control unit comprises hardware and software for controlling the infusion device 3. The software comprises a plurality of instructions for running an algorithm designed to calculate the flow rate as dictated by the flow rate delivery function. In some embodiments, the flow rate delivery function characterises a flow rate at which the pharmaceutical preparation is provided, by the medication delivery apparatus 2, to the patient. FIG. 1B show a block diagram of the medication delivery system 1.

The medication delivery apparatus 2 comprises a first plunger 13. The first plunger 13 may also be referred to as a primary plunger. The medication delivery apparatus 2 comprises a second plunger 14. The second plunger 14 may also be referred to as a separating plunger. The medication delivery apparatus 2 comprises a container 11. The container 11 may also be referred to as a barrel 11. The container 11 is configured to receiving the second plunger 14. The container 11 is configured to receive at least a portion of the first plunger 13. This may be a distal portion of the first plunger 13.

The second plunger 14, when received within the container 11, defines two chambers within the container 11. In particular, the second plunger 14, when received within the container 11, defines a first chamber 15 and a second chamber 25. The first chamber 15 is configured to store a first fluid. The first fluid may be a solution comprising an active agent and a solvent. The active agent may be as described herein. The solvent may be as described herein. The first chamber 15 may be referred to as an active agent chamber 15. The second chamber 25 is configured to store a second fluid. The second fluid may be a diluent. The diluent may be as is described herein. The second chamber 25 may be referred to as mixing chamber 25 or a dilution chamber 25. In particular, the container 11 and the second plunger 14 together define a dilution chamber 25. The dilution chamber 25 is configured to receive a diluent. The first plunger 13, the container 11 and the second plunger 14 together define the active agent chamber 15.

The active agent chamber 15 is configured to receive an active agent. In some embodiments, the active agent may be a solid (e.g. in crystalline form or a powder). In such cases, the active agent chamber 15 may be configured to receive a solvent. The solvent is configured to dissolve the solid active agent. The pharmaceutical preparation therefore comprises the active agent dissolved in the solvent. That is, the pharmaceutical preparation comprises a solution comprising the active agent and the solvent.

In some embodiments, the active agent chamber 15 is configured to receive the pharmaceutical preparation. As described herein, the pharmaceutical preparation may be a solution comprising the active agent (when dissolved) and the solvent. In these cases, the active agent chamber 15 is configured to receive the pharmaceutical preparation as a solution comprising the active agent and the solvent.

The second plunger 14 is configured to enable flow of a fluid (e.g. the pharmaceutical preparation) contained in the active agent chamber 15 into the dilution chamber 25. The dilution chamber 25 comprises the diluent for mixing with the pharmaceutical preparation (or active agent) flowing from the active agent chamber 15.

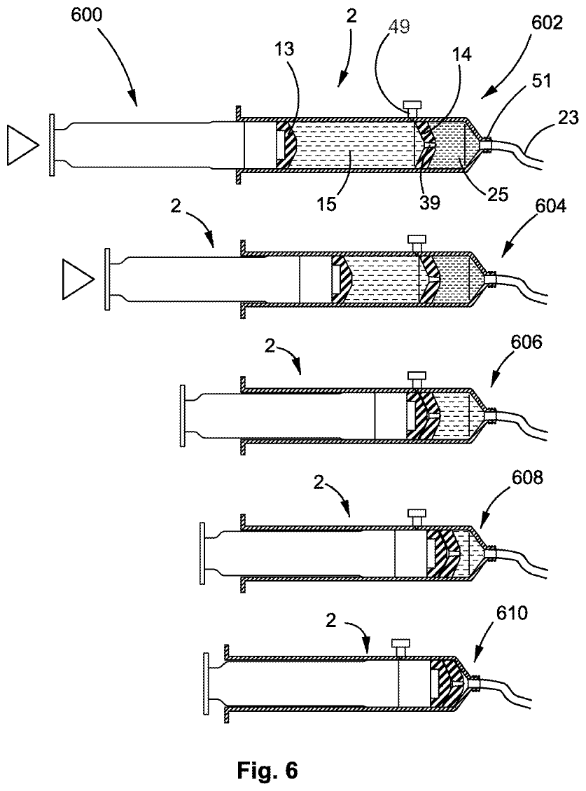

The second plunger 14 comprises a valve 39. The valve 39 may also be referred to as a valve means 39. The valve 39 is configured to control flow the pharmaceutical preparation entering the dilution chamber 25 from the active agent chamber 15. In other words, the second plunger 14 comprises a valve 39 configured to control a flow of pharmaceutical preparation from the active agent chamber 15 to the dilution chamber 25. The dilution chamber 25 is proximate the distal end of the container 11, while the active agent chamber 15 is further away from the distal end of the container. As shown in FIG. 6, in use pharmaceutical preparation may be expelled from the active agent chamber 15 through the valve 39 into the dilution chamber 25 and from the dilution chamber 25 out through the dilution chamber opening 51. The dilution chamber opening 51 may be connected to conduit 23, such as extension tubing, which leads to a patient. In this way pharmaceutical preparation may be mixed with the diluent in the dilution chamber and diluted pharmaceutical preparation may be ejected from the apparatus and delivered intravenously to a patient. As fluid may be continuously forced through the chambers and out of the dilution chamber opening, the mixing of the pharmaceutical preparation and the diluent in the dilution chamber may happen at the same time as diluted pharmaceutical preparation is ejected through the dilution chamber opening.

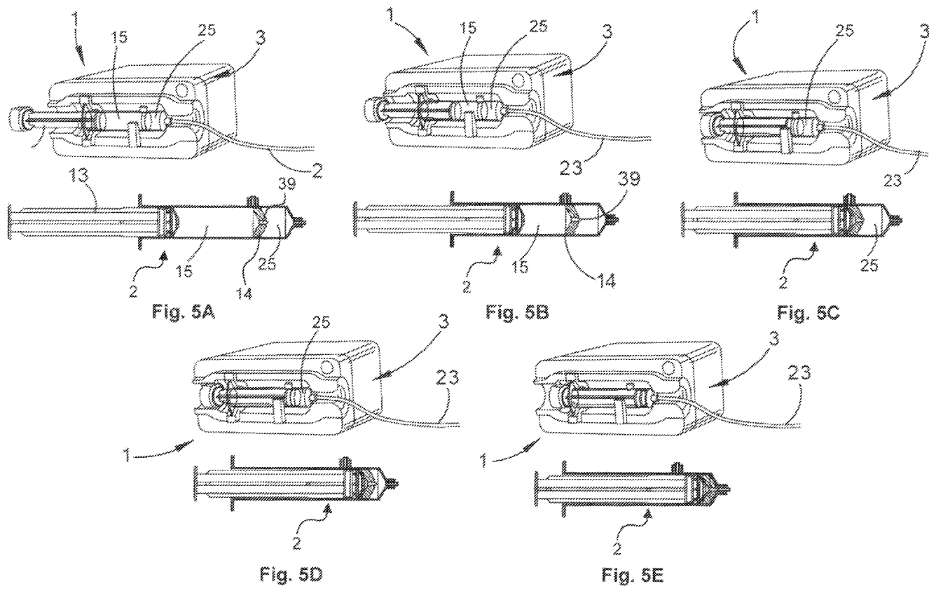

In FIGS. 5A-5D and FIG. 6, the dilution chamber 25 is towards the distal end of the container 11 and the active agent chamber 15 is towards the proximal end. This arrangement is different to prior art double chamber syringes in which a dilution chamber is at the proximal end and an active agent chamber at the distal end, because in the examples of the present disclosure the order of the chambers is different. In some examples, the medical delivery apparatus of the present disclosure, may be provided with empty dilution and active agent chambers which are to be filled by the clinician. This is different to prior art double chamber syringes in which the syringe is provided with an active agent chamber at the distal end pre-filled with a solid drug and a dilution chamber at the proximal end pre-filled with a diluent.

The valve 39 is a one-way valve, which allows flow of fluid from the activate agent chamber 15 to the dilution chamber 25, but inhibits flow of fluid from the dilution chamber into the active agent chamber. The valve 39 may be configured to control the flow of the pharmaceutical preparation in response to applied pressure. The pressure may be applied by the first plunger 13. Alternatively, the pressure may be applied via the first plunger 13. In the particular arrangement shown in at least FIGS. 1 to 8, the valve 39 comprises a duckbill valve 41. The duckbill valve 41 comprises a plurality of flaps 43 that, as pressure is applied to the first plunger 13, separate with respect to each other opening the duckbill valve 41. Upon removal of the pressure, that is being applied to the first plunger 13, the flaps 43 return to their original condition closing the duckbill valve 41 and impeding backflow of the pharmaceutical preparation back into the active agent chamber 15.

The valve 39 (or valve means 39) comprises an inlet side 45 and an outlet side 47. The valve 39 (or valve means 39) is configured to move from a closed position to an open position upon application of pressure to the inlet side 45. Pressure may be applied to the inlet side 45 of the valve 39 (or valve means 39) by longitudinally displacing (or actuating) the first plunger 13 within the container 11 to displace the pharmaceutical preparation. The valve 39 (or valve means 39) is configured to move from the open position to the closed position upon removal of the pressure applied to the inlet side. The valve 39 (or valve means 39) may be configured to move from the closed position to the open position when a pressure applied to the inlet side 45 exceeds a pressure threshold. The valve 39 (or valve means 39) may be configured to move from the open position to the closed position when the pressure applied to the inlet side 45 is below a pressure threshold.

The valve 39 (or valve means 39) is biased towards the closed position. The valve 39 (or valve means 39) comprises the plurality of flaps 43. The plurality of flaps 43 are configured to separate upon application of pressure to the inlet side 45. The first plunger 13 is configured to contact the second plunger 14 once all, or most of, the pharmaceutical preparation in the active agent chamber 15 has been transferred to the dilution chamber 25. Further actuation of the first plunger 13 will also result in movement of the second plunger 14. Thus, actuation of the first plunger 13 causes movement of the second plunger 14, and causes the pharmaceutical preparation in the dilution chamber 25 to be output by the medication delivery apparatus 2.

The container 11 comprises at least one first port 49. The first port 49 may be referred to as a container fill port 49. The first port 49 may be referred to as an active agent chamber port 49. This may be cause the first port 49 can provide access to the active agent chamber 15 of the medication delivery apparatus 2. The active agent chamber port 49 defines an active agent chamber port opening 54. The active agent chamber port opening 17 is an opening in the container 11 through which the active agent chamber 15 and/or an interior of the container 11 can be accessed. The medication delivery system 1 comprises a first port cap 50. The first port cap 50 is configured to cap the active agent chamber port 49. The first port cap 50 may be referred to as an active agent chamber port cap 50.

The container comprises a second port 51. The second port 51 may be referred to as a container outlet port 51. The second port 51 may also be referred to as a dilution chamber port 51. The dilution chamber port 51 defines a dilution chamber opening 53. The dilution chamber opening 53 is an opening in the container 11 through which the dilution chamber 25 and/or an interior of the container 11 can be accessed. The medication delivery system 1 comprises a second port cap 52. The second port cap 52 may be referred to as a container outlet port cap 52. The second port cap 52 may be referred to as a dilution chamber port cap 52. The dilution chamber port cap 52 is configured to cap the dilution chamber port 51.

The container fill port 49 enables filling of the container 11 with the pharmaceutical preparation. The dilution chamber port 51 enables either (1) filing of the dilution chamber 25 with diluent or (2) permitting exit of the mixture of the active agent and diluent (the pharmaceutical composition) from the container 11 (in particular, from the dilution chamber 25) for delivery to the patient.

As previously described, the container 11 comprises the active agent chamber opening 17. The active agent chamber opening 17 is configured to receive at least a portion of the first plunger 13. In particular, the active agent chamber 15 comprises the active agent chamber opening 17. The active agent chamber port opening 49 may be considered a second active agent chamber opening. In other words, the active agent chamber 15 may be said to comprise a second active agent chamber opening that is configured to receive the pharmaceutical preparation. The active agent chamber port opening 49 is defined in the wall of the container 11. The active agent chamber 15 may be filled with pharmaceutical preparation by introducing the pharmaceutical preparation into the active agent chamber 15 via the second active agent chamber port opening 49. The active agent chamber port 49 may therefore be referred to as an active agent chamber inlet.

As previously described, the container 11 comprises the dilution chamber port 51. In particular, the dilution chamber 25 comprises the dilution chamber port 51. The second port 51 may therefore be referred to as a dilution chamber port 51. The dilution chamber port 51 comprises a dilution chamber opening 53. The dilution chamber port 51 may therefore be considered an outlet port 51 of the medication delivery apparatus 2 and/or of the dilution chamber 25.

In the arrangement shown in figures, the active agent chamber port 49 and the dilution chamber port 51 are shown to comprise male Luer-lock connectors (i.e. a first Luer-lock connector). In alterative arrangements, for example, the active agent chamber port 49 and/or the dilution chamber port 51 may comprise a female Luer-lock connector (i.e. a second Luer-lock connector).

The active agent chamber port cap 50 may comprise a complementary Luer-lock connector to the Luer-lock connector of the active agent chamber port 49. The active agent chamber port cap 50 inhibits fluid flow through the active agent chamber port 49 while connected to the active agent chamber port 49. The dilution chamber port cap 52 may comprise a complementary Luer-lock connector to the Luer-lock connector of the dilution chamber port 51. The dilution chamber port cap 52 inhibits fluid flow through the dilution chamber port 51 while connected to the dilution chamber port 51.

The first plunger 13 and the second plunger 14 are each configured to be displaced with respect to a longitudinal axis 21 of the container 11. The second plunger 14 is disposed between the first plunger 13 and the dilution chamber opening 53 (and the dilution chamber port 51). The second plunger 14 is disposed between the active agent chamber port 49 (and the active agent chamber port opening) and the dilution chamber opening 53.

The container 11 defines a inner container surface 55. The first plunger 13 comprises a first plunger sealing surface 57. The first plunger 13 is configured to seal with the inner container surface 55. In particular, the first plunger 13 is configured to seal with the inner container surface 55 to inhibit fluid flow between the inner container surface 55 and the first plunger sealing surface 57. The first plunger 13 is configured to seal with the first plunger sealing surface 57.

The second plunger 14 comprises a second plunger sealing surface 59. The second plunger 14 is configured to seal with the inner container surface 55. In particular, the second plunger sealing surface 59 is configured to seal with the inner container surface 55 to inhibit fluid flow between the inner container surface 55 and the second plunger sealing surface 59. The second plunger 14 is configured to seal with the second plunger sealing surface 59.

The medication delivery apparatus 2 may comprise a conduit 23. The conduit 23 is configured to be fluidly connected to the dilution chamber opening 53. The conduit 23 is of a predetermined volume. That is, a length and an internal surface area of the conduit 23 are sized so that the conduit 23 defines a predetermined volume. The conduit 23 can therefore hold or store a volume of the diluted pharmaceutical preparation prior to the diluted pharmaceutical preparation being delivered to the patient. The conduit 23 may be referred to as a minimum volume extension tube. The conduit 23 is configured to retain a first volume of infusion to be delivered to the patient. The first volume of infusion can be prepared by the priming process at a rate that will result in effective mixing in the dilution chamber 25. This is possible because during this time, no pharmaceutical preparation is delivered to the patient. Thus, a different flow rate can be used for the first volume when priming, while driving the mixed fluid exiting the dilution chamber 25 to the end of the conduit 23. Although the conduit 23 of the medication delivery apparatus 2 is described to be of a predetermined volume, it will be understood that a conduit of a predetermined volume could be used with any of the medication delivery apparatuses disclosed herein to achieve similar functionality and benefits.

The infusion device 3 comprises a computer system 5. The infusion device 3 comprises a driving mechanism 9. The drive mechanism 9 may comprise a syringe driver. The driving mechanism 9 is controlled by the computer system 5. In particular, the computer system 5 is adapted to control the driving mechanism 9 in order to deliver the drug (contained in the medication delivery apparatus 2) to the patient in a specific manner, for example, in accordance with the flow rate delivery function.

The computer system 5 comprises at least one processor 27. The computer system 5 comprises memory 29. Memory 29 may be in the form of random access memory (RAM). The computer system 5 comprises a data storage device 31. The computer system 5 comprises a user interface 33. The user interface 33 may comprise a display 35 and/or a keyboard 37. A particular component of the computer system 5 may communicate with one or more other components of the computer system 5 and/or the infusion device 3 via a system bus 39.

The at least one processor 27 is configured to execute infusion device program instructions stored in the memory 29 to cause the infusion device 3 to function as described herein. In other words, the infusion device program instructions are accessible by the at least one processor 27, and are configured to cause the at least one processor 27 to function as described herein.

In some embodiments, the infusion device program instructions are in the form of program code. The at least one processor 27 comprises one or more microprocessors, central processing units (CPUs), application specific instruction set processors (ASIPs), application specific integrated circuits (ASICs) or other processors capable of reading and executing program code.

The memory 29 may comprise one or more volatile or non-volatile memory types. For example, the memory 29 may comprise one or more of random access memory (RAM), read-only memory (ROM), electrically erasable programmable read-only memory (EEPROM) or flash memory. The memory 29 is configured to store program code accessible by the at least one processor 27. The program code may comprise executable program code modules. In other words, memory 29 is configured to store executable code modules configured to be executable by the at least one processor 27. The executable code modules, when executed by the at least one processor 27 cause the at least one infusion device processor 27 to perform certain functionality, as described herein.

Where the infusion device 3 comprises or is in the form of the vacuum infusion device 3, the infusion device 3 may apply an infusion pressure (i.e. the vacuum pressure 61) at the dilution chamber opening, thereby causing displacement of the first plunger 13 such that the pharmaceutical preparation is output by the medication delivery apparatus 2 at a target flow rate.

In some embodiments, the infusion device 3 is configured to actuate the first plunger 13. The infusion device 3 may be configured to actuate an infusion device actuator to displace the first plunger 13 such that the pharmaceutical preparation is output by the medication delivery apparatus 2. The pharmaceutical preparation may be output at a target flow rate.

The computer system 5 may optionally include a drug library, and database which contains the maximum allowable drug administration rate for each particular drug that may be infused to patients. If the drug delivery rate expected during use of the infusion device 3 (e.g. during execution of the flow rate delivery function), exceeds the maximum allowable drug administration rate, then the infusion rate will be reduced according to the maximum allowed infusion rate such that the concentration of drug leaving the dilution chamber 25 does not exceed the maximum allowable drug administration rate. This may result in the infusion time being greater than intended for the infusion, but ensures that the maximum permitted or suggested pharmaceutical drug administration rate is not exceeded.

During the method of infusing the pharmaceutical preparation in accordance with the present methods of the disclosure, the drug library may be accessed by the computer system 5 to confirm whether the drug delivery rate exceeds the maximum allowable drug administration rate; and if it does then, the infusion rate will be reduced according to the maximum allowed infusion rate to give the maximum allowable drug administration rate.

The processor 27 may execute instructions to control the driving mechanism 9 of the infusion device 3 in order to deliver the drug in accordance to, for example, the flow rate delivery function. The code executed by the processor 27 may be stored in the memory 29 of the computer system 5 or may be provided from external sources through the data storage device 31. This software will include the instructions to control the driving mechanism of the infusion device 3 such that the pharmaceutical preparation exits the medication delivery apparatus 2 at a particular flow rate to match, or approximate the infusion rate of the pharmaceutical preparation dictated by the flow rate delivery function.

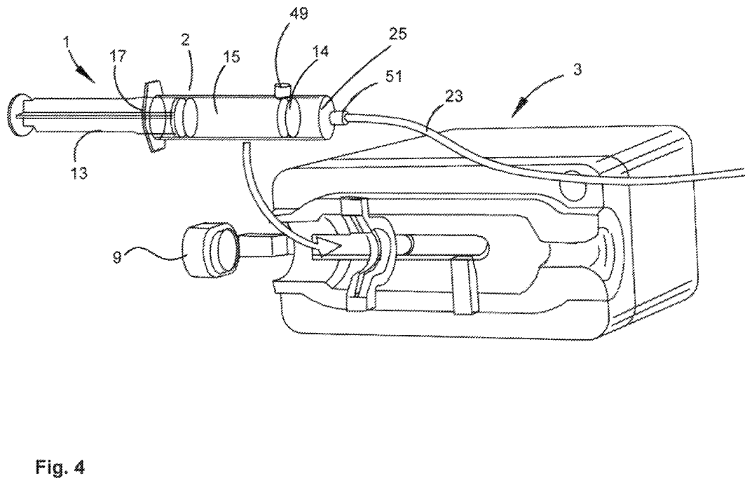

FIGS. 4 and 5A to 5E illustrate the medication delivery apparatus 2 mounted on the infusion device 3, thereby forming a medication delivery system 1. The infusion device 3 of FIGS. 4 and 5A to 5E is in the form of a syringe driver 3. FIG. 6 illustrates a first method 600 of operation of the medication delivery apparatus 2, according to some embodiments. The first method 600 of operation of the medication delivery apparatus 2 in FIGS. 5A to 6 comprises actuating the first plunger 13 by applying a force on the first plunger in a direction parallel to the longitudinal axis 21 of the medication delivery apparatus 2. The force is applied by the infusion driver 9, which contacts and pushes the first plunger 13. FIGS. 5A to 6 illustrate a number of steps involved in evacuating the active agent chamber 15 and the dilution chamber 25 in accordance with the first method 600.

As shown in FIG. 5A to 5E, preparation of the pharmaceutical composition (i.e. the solution output by the medication delivery apparatus 2) comprises the step of pushing the first plunger 13 so that the pharmaceutical preparation contained in the active agent chamber 15 is delivered into the dilution chamber 25 for mixing with the diluent contained in the diluent chamber 25 (via the valve 39). In FIG. 6, this step is shown at 602 and 604.

At 602, the medication delivery apparatus 2 is in a primed state. In the primed state, the active agent chamber 15 is filled with the pharmaceutical preparation, the dilution chamber 25 is filled with the diluent and the conduit 23 is primed with diluent.

In FIGS. 5A to 5C and at 602 and 604 in FIG. 6, the infusion device 3 engages and actuates the first plunger 13. The infusion device 3 actuates the first plunger 13 in a direction that is parallel to the longitudinal axis 21 of the medication delivery apparatus 2. In FIGS. 5A to 5C and at 604, the primary plunger 13 is pushed by the infusion device 3 so that the pharmaceutical preparation is delivered into the dilution chamber 25 in order to provide, in conjunction with the valve means 39, a particular mixing profile within the dilution chamber 25 to allow proper mixing of the pharmaceutical preparation with the diluent.

As the pharmaceutical preparation contained in the active agent chamber 15 is delivered into the dilution chamber 25, mixing occurs for generating the pharmaceutical composition (in this case, the diluted pharmaceutical preparation), which is then delivered into the conduit 23 for infusion into the patient. As the pharmaceutical composition is delivered into the conduit 23, the concentration of active agent within the dilution chamber 25 will increase as the pharmaceutical preparation is delivered into the dilution chamber 25 during the infusion.

In FIGS. 5D and 5E and at 606 to 610 of FIG. 6, the active agent chamber 15 has been evacuated. That is, the first plunger 13 has contacted the second plunger 14. Further actuation of the first plunger 13 causes motion of the second plunger 14. The motion of the second plunger 14 causes the fluid in the dilution chamber 25 (i.e. the diluted pharmaceutical preparation, or pharmaceutical composition) to be displaced through the dilution chamber port 51. The fluid in the dilution chamber 25 is displaced into the conduit 23 for provision to the patient.

For delivery of the pharmaceutical composition to the patient, the first plunger 13 (with the second plunger 14 abutting the primary plunger 13) is pushed in such a manner that the pharmaceutical composition is delivered in accordance with a particular profile. In particular, the first plunger 13 is driven based on particular algorithms.

Initially, before the first plunger 13 is driven based on the particular algorithms and the conduit 23 is fluidly connected to the patient, the infusion device 3 is operated to drive the first plunger 13 in such a manner to fill (i.e. to prime) the conduit 23 to be fluidly connected to the patient for delivery of the pharmaceutical composition.

Alternatively, in some embodiments, before the first plunger 13 is driven based on the particular algorithms and the conduit 23 is fluidly connected to the patient, the conduit 23 is filled with diluent. The infusion device 3 may then deliver the volume required to displace the volume of diluent in the conduit 23 into the patient and fill the conduit 23 with diluted pharmaceutical preparation before the first plunger 13 is driven based on the particular algorithms.

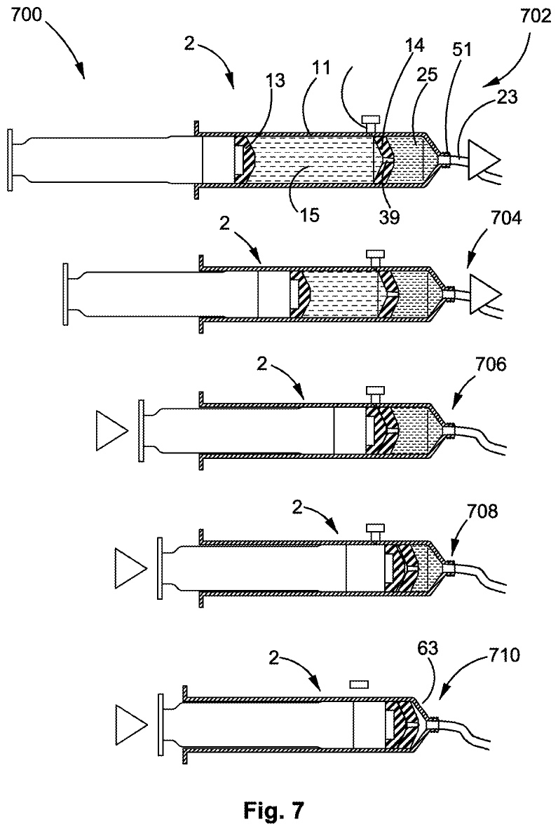

FIG. 7 illustrates a second method 700 of operation of the medication delivery apparatus 2, according to some embodiments. The second method 700 of operation of the medication delivery apparatus 2 in comprises actuating the first plunger 13 by applying a vacuum pressure 61 to dilution chamber port 51. The force may be applied by a vacuum pump of the infusion driver 3. The vacuum pressure 61 applies a force on the first plunger 13. The force causes the first plunger 13 to move in a direction parallel to the longitudinal axis 21 of the medication delivery apparatus 2, towards the second plunger 14. FIG. 7 illustrates the number of steps involved in evacuating the active agent chamber 15 and the dilution chamber 25 in accordance with the second method 700.

At 702, the medication delivery apparatus is in a primed state. In the primed state, the active agent chamber 15 is filled with the pharmaceutical preparation, the dilution chamber 25 is filled with the diluent and the conduit 23 is primed with diluent.

At 704, the infusion device 3 applies the vacuum pressure 61 to the dilution chamber port 51. The vacuum pressure 61 may be applied via the conduit 23. As is described herein, the medication delivery apparatus 2 is configured such that the first plunger 13 is moved by the vacuum pressure 61. The first plunger 13 is, initially, moved by the vacuum pressure 61 without significant movement of the second plunger 14. At 704, the first plunger 13 moves, thereby displacing pharmaceutical preparation from the active agent chamber 15 into the dilution chamber 25 via the valve 39. The movement of the first plunger 13 also displaces the pharmaceutical composition (i.e. the diluted pharmaceutical preparation in the dilution chamber 25) through the dilution chamber port 51. The medication delivery apparatus 2 is shown at 704 in a first intermediate state. In the first intermediate state, the first plunger 13 has partially evacuated the pharmaceutical preparation from the active agent chamber 15.

At 706, the first plunger 13 contacts the second plunger 14. The infusion device 3 continues to apply the vacuum pressure 61, which causes movement of both the first plunger 13 and the second plunger 14. The movement of the first plunger 13 and the second plunger 14 displaces the fluid in the dilution chamber 25 (the pharmaceutical composition) through the dilution chamber port 51.

At 708, the first plunger 13 and the second plunger 14 are both moved by the vacuum pressure 61. The medication delivery apparatus 2 is shown at 708 in a second intermediate state. In the second intermediate state, the first plunger 13 and the second plunger 14 have partially evacuated the pharmaceutical composition from the dilution chamber 25.

At 710, the second plunger 14 contacts the container 11. In particular, the second plunger 14 contacts a distal end 63 of the container 11. At this point, both the active agent chamber 15 and the dilution chamber 25 have been emptied.

### Method 800 for Preparing the Medication Delivery Apparatus 2

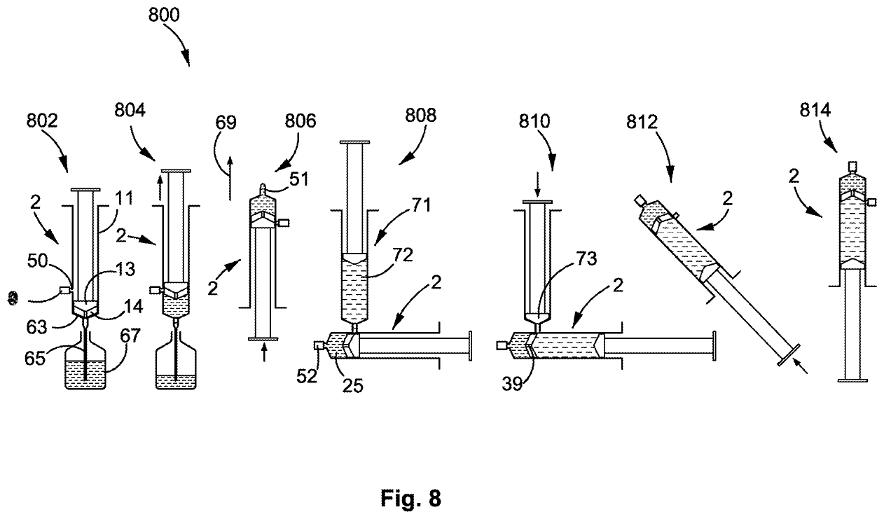

FIG. 8 illustrates a method 800 for preparing the medication delivery apparatus 2 of FIGS. 1 to 5E, according to some embodiments. At 802, the medication delivery apparatus 2 is in an initial state. In the initial state, the first plunger 13 is in contact with the second plunger 14. Furthermore, the second plunger is in contact with the distal end 63 of the container 11.

The clinician caps the active agent chamber port 49 with the active agent chamber port cap 50. The clinician connects a filling conduit 65 to the dilution chamber port 51 of the medication delivery apparatus 2. The filling conduit 65 may comprise a complementary Luer-lock connector to the Luer-lock connector of the dilution chamber port 51. The filling conduit 65 may be in the form of a needle.

The clinician inserts a distal end of the filling conduit 65 into a diluent container 67. The diluent container 67 comprises the diluent. The clinician inserts the distal end of the filling conduit 65 into the diluent that is stored in the diluent container 67.

At 804, the clinician moves the first plunger 13 away from the dilution chamber port 51. Moving the first plunger 13 away from the dilution chamber port 51 draws second plunger 14 away from the dilution chamber port 51 with the first plunger 13 as there is a substantially airtight seal between the first plunger 13 and the second plunger 14. Moving the first plunger 13 away from the dilution chamber port 51 also draws the diluent into the medication delivery apparatus 2 from the diluent container 67.

Moving the first plunger 13 away from the dilution chamber port 51 increases a volume of the dilution chamber 25. The increased dilution chamber 25 volume is occupied by the diluent that is drawn through the second outlet 51. The clinician moves the first plunger 13 (and the second plunger 14) away from the dilution chamber port 51 to an initial dilution chamber plunger position. The dilution chamber 25 is an initial dilution chamber volume when the first plunger 13 is in the initial dilution chamber plunger position. The initial dilution chamber volume may relate to a volume of diluent that is to be provided to the patient during an infusion. For example, the initial dilution chamber volume may be larger than the volume of diluent that is to be provided to the patient during an infusion.

At 806, the clinician removes the filling conduit 65 from the dilution chamber port 51. The clinician moves the medication delivery apparatus 2 so that the dilution chamber port 51 is facing an upwards direction 69. The clinician moves the medication delivery apparatus 2 (e.g. by tapping the medication delivery apparatus 2) to cause bubbles that may be in the diluent within the dilution chamber 25 to move towards the dilution chamber port 51. The clinician then moves the first plunger 13 (and the second plunger 14) towards the dilution chamber port 51 to a dilution chamber plunger position, thereby forcing excess air out of the dilution chamber port 51. The dilution chamber 25 is a dilution chamber infusion volume when the first plunger 13 is in the dilution chamber plunger position. The dilution chamber infusion volume may relate to a volume of diluent that is to be provided to the patient during an infusion. For example, the dilution chamber infusion volume may correspond to the volume of diluent that is to be provided to the patient during an infusion.

At 808, the clinician caps the dilution chamber port 51 with the dilution chamber port cap 52. The dilution chamber port cap 52 may comprise a complementary Luer-lock connector to the Luer-lock connector of the dilution chamber port 51. The dilution chamber port cap 52 inhibits fluid flow through the dilution chamber port 51 while connected to the dilution chamber port 51.

The clinician removes the active agent chamber port cap 50 from the active agent chamber port 49. The clinician connects an outlet of a filling syringe 71 to the active agent chamber port 49. The filling syringe 71 contains the pharmaceutical preparation that is to be used in the infusion in a filling syringe chamber 72.

At 810, the clinician actuates a filling syringe plunger 73 of the filling syringe 71. Actuation of the filling syringe plunger 73 displaces the pharmaceutical preparation through the outlet of the filling syringe 71 and into the medication delivery apparatus 2. The pharmaceutical preparation applies a pressure on the first plunger 13 and causes displacement of the first plunger 13 within the container 11. The pressure applied by the pharmaceutical preparation moves the first plunger 13 away from the second plunger 14.

Alternatively, the clinician can move the first plunger 13 away from the second plunger 14, increasing the volume of the active agent chamber 15. This creates a vacuum pressure at the outlet of the filling syringe 71 causing pharmaceutical preparation to be displaced from the filling syringe 71 and into the active agent chamber 15.

Moving the first plunger 13 away from the second plunger 14 increases a volume of the active agent chamber 15. The increased active agent chamber 15 volume is occupied by the pharmaceutical preparation. The first plunger 13 is moved to an initial active agent chamber first plunger position. The active agent chamber 15 is an initial active agent chamber volume when the first plunger 13 is in the initial active agent chamber first plunger position. The initial active agent chamber volume may relate to a volume of pharmaceutical preparation that is to be provided to the patient during the infusion. For example, the initial active agent chamber volume may be larger than the volume of pharmaceutical preparation that is to be provided to the patient during an infusion.

At 812, the clinician removes the filling syringe 71. The clinician moves the medication delivery apparatus 2 so that the first port 51 is generally facing the upwards direction 69. The clinician moves the medication delivery apparatus 2 (e.g. by tapping the medication delivery apparatus 2) to cause bubbles that may be in the pharmaceutical preparation within the active agent chamber 15 to move towards the active agent chamber port 49. The clinician then moves the first plunger 13 towards the active agent chamber port 49 to an active agent chamber plunger position, thereby forcing air remaining in the active agent chamber 15 out through the active agent chamber port 49. The active agent chamber 15 is an active agent chamber infusion volume when the first plunger 13 is in the active agent chamber plunger position. The active agent chamber infusion volume may relate to a volume of pharmaceutical preparation that is to be provided to the patient during the infusion. For example, the active agent chamber infusion volume may correspond to the volume of the pharmaceutical preparation that is to be provided to the patient during the infusion.

At 814, the clinician caps the active agent chamber port 49 with the active agent chamber port cap 50. The medication delivery apparatus 2 may now be stored for a later infusion, or used in an infusion.

### Method 900 for Preparing the Medication Delivery Apparatus 2

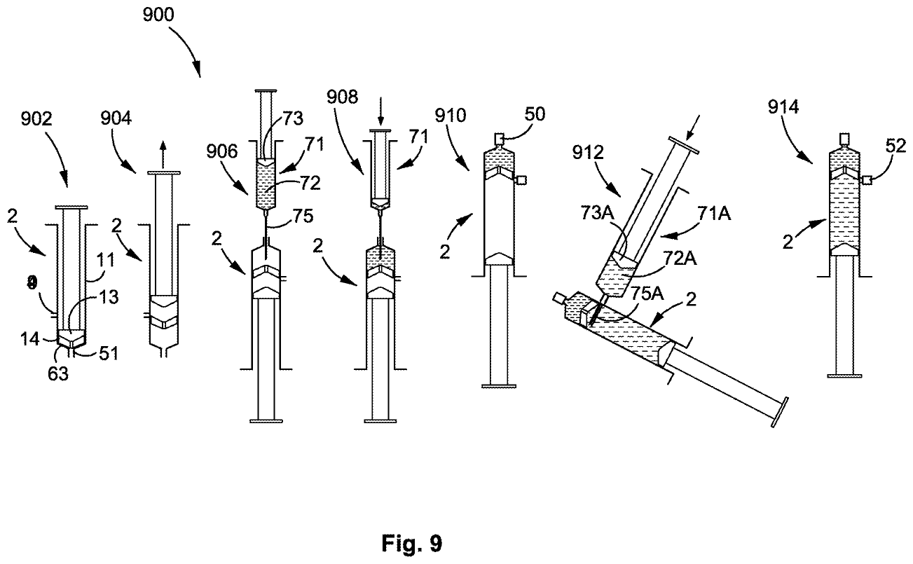

FIG. 9 illustrates a method 900 of preparing the medication delivery apparatus 2 of FIGS. 1 to 5E, according to some embodiments. At 902, the medication delivery apparatus 2 is in an initial state. In the initial state, the first plunger 13 is in contact with the second plunger 14. Furthermore, the second plunger 14 is in contact with the distal end 63 of the container 11. In the initial state, the active agent chamber port cap 50 and the dilution chamber port cap 52 are not connected to the medication delivery apparatus 2.

At 904, the clinician moves the first plunger 13 away from the dilution chamber port 51. Moving the first plunger 13 away from the dilution chamber port 51 draws second plunger 14 away from the dilution chamber port 51 with the first plunger 13 as there is a substantially airtight seal between the first plunger 13 and the second plunger 14. The clinician draws the first plunger 13 away from the dilution chamber port 51 and past the active agent chamber port 49.

When the first plunger 13 is moved past the active agent chamber port 49, the substantially airtight seal between the first plunger 13 and the second plunger 14 is broken by the active agent chamber port 49. That is, air is able to enter the medication delivery apparatus between the first plunger 13 and the second plunger 14 via the active agent chamber port opening 54. The second plunger 14 therefore stops moving with the first plunger 13 as the first plunger 13 is moved further away from the dilution chamber port 51. The second plunger 14 is left in a dilution chamber plunger position.

At 906 and 908, the clinician uses a first filling syringe 71 to fill the dilution chamber 25 with the diluent. The first filling syringe 71 comprises a first filling syringe chamber 72 that comprises the diluent. The first filling syringe 71 comprises a first filling syringe conduit 75 which may be, for example, a needle. At 906, the clinician inserts the first filling syringe conduit 75 in the dilution chamber port 51.

At 908, the clinician actuates a first filling syringe plunger 73 to displace the diluent from the first filling syringe chamber 72 through the first filling syringe conduit 75 and into the dilution chamber 25. An outer diameter of the first filling syringe conduit 75 is less than an inner diameter of the opening of the dilution chamber port 51. Thus, bubbles that are present in the diluent when displaced into the dilution chamber 25 can flow past the first filling syringe conduit 75 and out of the dilution chamber 25 during filling.

At 910, the clinician caps the second outlet 51 with the dilution chamber port cap 52. The clinician then moves the first plunger 13 to an active agent chamber plunger position. The active agent chamber plunger position corresponds to a required pharmaceutical preparation volume (i.e. a required capacity of the active agent chamber 15).

At 912, the clinician uses a second filling syringe 71A to fill the active agent chamber 15 with the pharmaceutical preparation. The second filling syringe 71A comprises a second filling syringe chamber 72A that comprises the pharmaceutical preparation. The second filling syringe 71A comprises a second filling syringe conduit 75A which may be, for example, a needle. The clinician inserts the second filling syringe conduit 75A in the active agent chamber port 49.

The clinician actuates a second filling syringe plunger 73A to displace the pharmaceutical preparation from the second filling syringe chamber 72A through the second filling syringe conduit 75A and into the active agent chamber 15. An outer diameter of the second filling syringe conduit 75A is less than an inner diameter of the active agent chamber port opening 54. Thus, bubbles that are present in the pharmaceutical preparation when displaced into the active agent chamber 15 can flow past the second filling syringe conduit 75A and out of the active agent chamber port opening 54 during filling.

At 914, the clinician caps the active agent chamber port 49 with the active agent chamber port cap 50. The medication delivery apparatus 2 is thereby in a storage state, and can be stored for a later infusion, or used in an infusion.

### Medication Delivery Apparatus 2 Comprising a Concave First Plunger and Bi-Convex Second Plunger

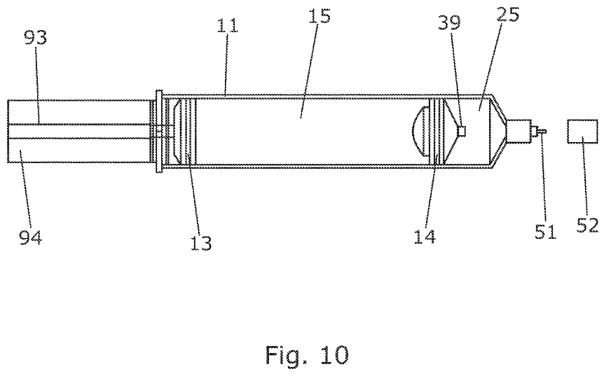

FIG. 10 illustrates an example of a medication delivery apparatus 2 in which the first plunger and second plunger have a particular shape. Like reference numerals denote like parts as in the previous figures. The first plunger 13 has a concave surface 13A facing the second plunger. The second plunger 14 may be described as bi-convex, it has a convex surface 14A facing the first plunger and a convex surface 14B facing the distal end of the container and dilution chamber opening 51. The dilution chamber opening may be closed by attaching an end cap 52 to prevent fluid flowing out of the opening. In this example, the valve 39 projects outwardly from the second plunger 14. This may help to maintain a separation of the second plunger from the distal end of the container 11. In other examples, the valve 39 may not project outwardly. In still other examples, the valve 39 may be contained entirely inside the second plunger so as to protect the valve 39 from distortion through contact with the container walls or dilution chamber opening 51. The first plunger 13 may comprise a gasket portion and a shaft 94 extending back from the gasket portion to a proximal end of the container 11. The shaft 94 may have a lumen 93 for allowing flow of fluid through the first plunger 13 into the active agent chamber 11. This lumen 93 may for instance be used for filling the active agent chamber 15 with pharmaceutical preparation or aspirating air from the active agent chamber 15.

The facing surfaces 13A, 14A of the first plunger and the second plunger may at least partially conform to each other. This minimises wastage of pharmaceutical preparation by minimising or preventing pharmaceutical preparation getting trapped between the first and second plungers. As the surface 14B of the second plunger facing the dilution chamber opening 52 is concave this may help to minimise wastage of pharmaceutical preparation, especially if the internal surface of the distal end of the container 11 is convex, as this arrangement allows all or the majority of fluid to be expelled from the dilution chamber when the second plunger is moved into contact with the distal end of the container. A further example of a concave first plunger and bi-convex second plunger is described later in FIG. 26.

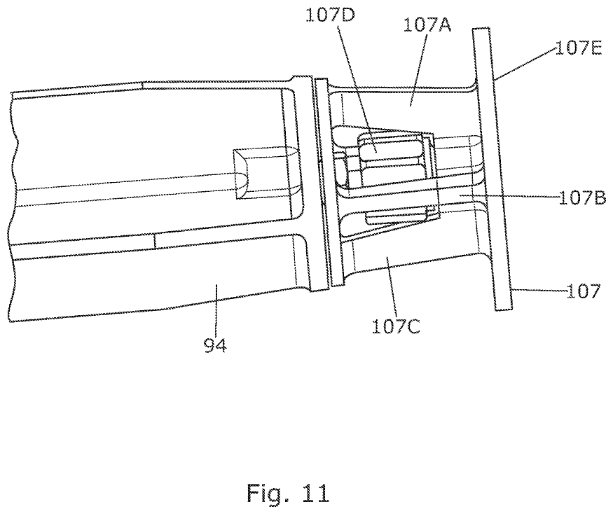

FIG. 11 shows an example of a cap 107 which may be attached to the distal end of the first plunger 13. As shown, the cap 107 is attached to the shaft 94 of the first plunger 13. The cap 107 may have a plurality of struts 107A-107C or other supporting structures to prevent bending of the cap 107 when the first plunger is pushed into the container 11. This helps to improve accuracy of the apparatus, as bending of the cap 107 can cause inaccuracy and make it difficult to move the first plunger to the desired position when filling the device or during an infusion. The cap 107 may further comprise a central portion 107D inside the struts, which central portion may screw onto the plunger lumen. The cap 107 may have an endplate 107E supported by the struts 107A-107C, which endplate may form a surface against which a syringe drive actuator, or a clinician, may push to move the first plunger 13 towards the distal end of the container 11.

### Medication Delivery Apparatus 2 Comprising a Plunger Lumen 93

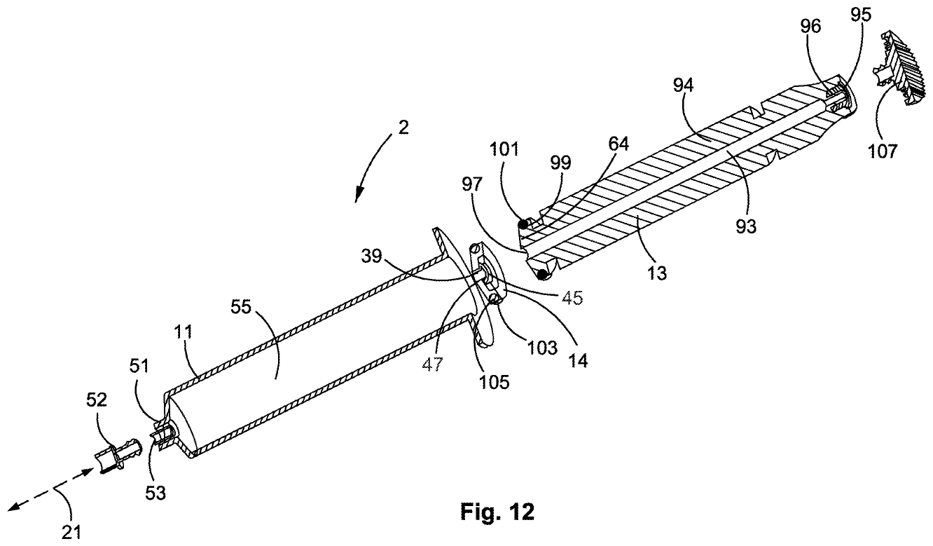

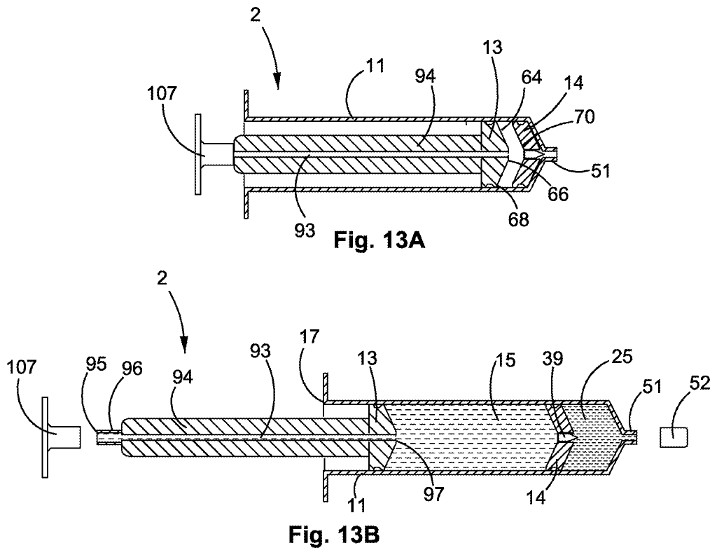

FIG. 12 illustrates a perspective view of a cross-section of another embodiment of the medication delivery apparatus 2, according to some embodiments. FIG. 13A is a schematic diagram of a cross section of the medication delivery apparatus 2 of FIG. 12, when empty. FIG. 13B is a schematic diagram of a cross section of the medication delivery apparatus 2 when filled with the diluent and the pharmaceutical preparation.

Referring to FIGS. 12, 13A and 13B, the medication delivery apparatus 2 comprises a number of features that are similar to, or the same as the other embodiments of the medication delivery apparatus 2 described herein. It will be understood that the medication delivery apparatus 2 of FIG. 12 may comprise features and/or components of other embodiments of the medication delivery apparatus 2 described herein.

The medication delivery apparatus 2 comprises a first plunger 13. The medication delivery apparatus 2 comprises a second plunger 14. The medication delivery apparatus 2 comprises a container 11. The container 11 is configured to receive the second plunger 14. The container 11 is configured to receive at least a portion of the first plunger 13. The container 11 and the second plunger define a dilution chamber 25 that is configured to receive a diluent. The dilution chamber 25 may be as described herein with reference to other embodiments of the medication delivery apparatus 2. The first plunger 13, the container 11 and the second plunger 14 define an active agent chamber 15. The active agent chamber 15 is configured to receive a pharmaceutical preparation. The active agent chamber 15 may be as described herein with reference to other embodiments of the medication delivery apparatus 2.

The container 11 comprises a dilution chamber port 51. The dilution chamber port 51 defines a dilution chamber opening 53. The dilution chamber port 51 and/or the dilution chamber opening 53 are configured to enable the flow of fluid into and out of the dilution chamber 25 and/or the container 11. The dilution chamber port 51 and/or the dilution chamber opening 53 may be the same as, or similar to the dilution chamber port 51 and/or the dilution chamber opening 53 described herein with reference to other embodiments of the medication delivery apparatus 2. The medication delivery apparatus 2 comprises a dilution chamber port cap 52. The dilution chamber port cap 52 may be the same as, or similar to the dilution chamber port cap 52 described herein with reference to other embodiments of the medication delivery apparatus 2. The dilution chamber port cap 52 is configured to connect to the container 11 to cap the dilution chamber opening 53.

The second plunger 2 comprises a valve 39. The valve 39 is configured to control a flow of pharmaceutical preparation from the active agent chamber 15 to the dilution chamber 25. The valve 39 may be the same as, or similar to the valve 39 described herein with reference to other embodiments of the medication delivery apparatus 2. For example, the valve 39 comprises a valve inlet side 45 and a valve outlet side 47. The valve 39 is configured to move from a closed position to an open position upon application of pressure to the inlet side 45, as described herein. The valve 39 is configured to move from the open position to the closed position upon removal of the pressure applied to the inlet side 45 as described herein.

The first plunger 13 comprises a plunger lumen 93. The plunger lumen 93 extends between a first plunger lumen opening 95 and a second plunger lumen opening 97. The plunger lumen 93 extends through a shaft 94 of the first plunger 13. The shaft 94 may also be referred to as a rod. In use, the plunger lumen 93 is generally parallel to the longitudinal axis 21 of the medication delivery apparatus 2. The active agent chamber 15 is configured to receive the pharmaceutical preparation through the plunger lumen 93.

The first plunger lumen opening 95 is a plunger lumen inlet. The first plunger lumen opening 95 is configured to receive pharmaceutical preparation that is to be provided through the plunger lumen 93. The second plunger lumen opening 97 is a plunger lumen outlet. The second plunger lumen opening 97 is configured to enable pharmaceutical preparation within the plunger lumen 93 to flow into the active agent chamber 15.

The first plunger 13 comprises a first plunger connector 96. The first plunger connector 96 is in the form of a first Luer-lock connector. The first plunger connector 96 defines the first plunger lumen opening 95.

The first plunger 13 comprises a first plunger O-ring 99. The first plunger O-ring 99 comprises a first plunger sealing surface 101. The first plunger 13 is configured to seal with the container 11 to provide a first seal. In particular, the first plunger sealing surface 101 is configured to seal with the inner container surface 55 to inhibit fluid flow between the inner container surface 55 and the first plunger sealing surface 101.

Alternatively, in some embodiments, the first plunger 13 comprises a first plunger sealing portion. The first plunger sealing portion may be an elastomeric portion. The first plunger sealing portion may be as described herein. The first plunger sealing portion may be configured to seal with the inner container surface 55 to inhibit fluid flow between the inner container surface 55 and the first plunger sealing surface 101. In some embodiments, the first plunger sealing portion comprises a projecting portion. The projecting portion may extend around a circumference of the first plunger 13. The projecting portion may be configured to seal with the inner container surface 55 to inhibit fluid flow between the inner container surface 55 and the first plunger sealing surface 101.

The first plunger 13 comprises a one-way valve (not shown). The one way valve may be disposed at a distal end 64 of the first plunger 13. For example, the one-way valve, when open, may define the second plunger lumen opening 97. The one-way valve advantageously enables fluid to flow through the plunger lumen 93 from the first plunger lumen opening 95 to the active agent chamber 15 without flowing in the opposite direction, once it has entered the active agent chamber 15.

The medication delivery apparatus 2 comprises a first plunger cap 107. The first plunger cap 107 is configured to connect to the first plunger 13. In particular, the first plunger cap 107 is configured to connect to the first plunger 13 to cap the first plunger lumen opening 95. The first plunger cap 107 is configured to connect to the first plunger connector 96. Thus, the first plunger cap 107 may comprise a complementary connector (e.g. a complementary Luer-lock connector) to that of the first plunger connector 96. In the illustrated embodiment, the first plunger cap 107 comprises a second Luer-lock connector configured to connect with a first Luer-lock connector of the first plunger 13 (i.e. the Luer-lock connector of the first plunger connector 96).

The second plunger 14 is disposed between the first plunger 13 and the dilution chamber opening 53. The second plunger 14 comprises a second plunger O-ring 103. The second plunger O-ring 103 comprises a second plunger sealing surface 105. The second plunger 14 is configured to seal with the container 11 to provide a second seal. In particular, the second plunger sealing surface 105 is configured to seal with the inner container surface 55 to inhibit fluid flow between the inner container surface 55 and the second plunger sealing surface 105.

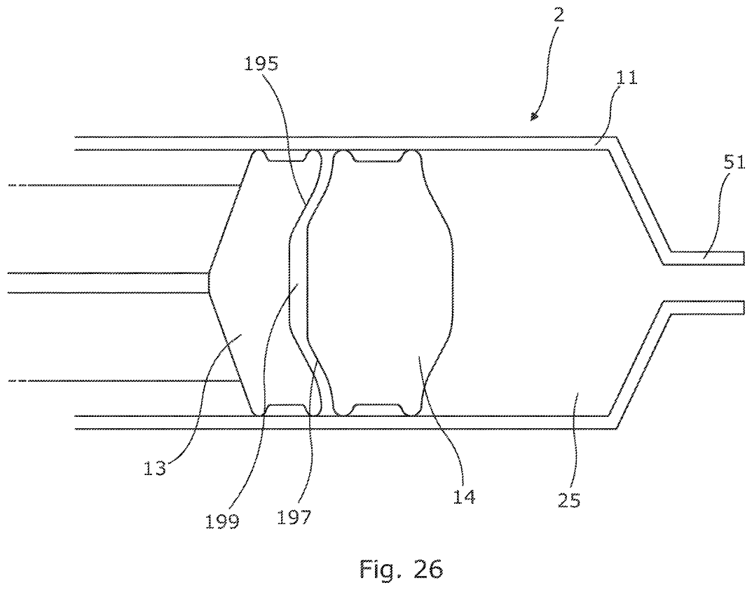

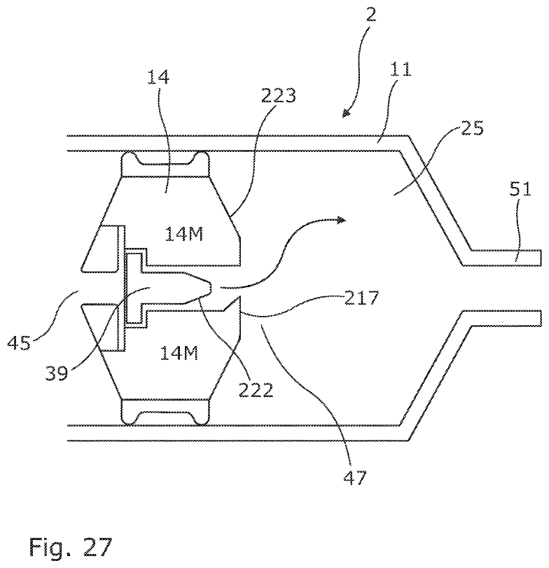

Alternatively, in some embodiments, the second plunger 14 comprises a second plunger sealing portion. The second plunger sealing portion may be an elastomeric portion. The second plunger sealing portion may be as described herein, for example, with reference to FIGS. 27A and 27B. The second plunger sealing portion may be configured to seal with the inner container surface 55 to inhibit fluid flow between the inner container surface 55 and the second plunger sealing surface 105. In some embodiments, the second plunger sealing portion comprises a projecting portion. The projecting portion may extend around a circumference of the second plunger 14. The projecting portion may be configured to seal with the inner container surface 55 to inhibit fluid flow between the inner container surface 55 and the second plunger sealing surface 105.

The first plunger 13 is configured to be displaced within the container 11 with respect to the longitudinal axis 21 of the container 11. The second plunger 14 is configured to be displaced within the container 11 with respect to the longitudinal axis 21 of the container 11.

A break loose force of the second plunger 14 is greater than a break loose force of the first plunger 13, as is described in more detail herein. A valve threshold force of the valve 39 is less than a sum of a break loose force of the first plunger 13 and a break loose force of the second plunger 14, as is described in more detail herein. For example, this approach may be used when the infusion device is a vacuum pump. In some embodiments, the valve threshold force is less than the break loose force of the second plunger 14. For example, this approach may be used when the infusion device is a syringe driver. The valve threshold force is the force required to open the valve. As the valve is part of the second plunger, when the valve is open and fluid is flowing through the valve, this flow of fluid will apply some force to the second plunger in the direction of the fluid flow. This force on the second plunger caused by flow of fluid through the open valve is referred to as the open valve force. The open valve force may be proportional to a resistance of the open valve. The apparatus may be designed such that the open valve force is less than the break loose force of the second plunger 14 even at high flow rates, so that the second plunger is not moved by high flow rates through the valve.

The medication delivery apparatus 2 comprises a conduit (not shown). The conduit is configured to be fluidly connected to the dilution chamber opening 53. The conduit is of a predetermined volume. The conduit of the medication delivery apparatus 2 illustrated with respect to FIG. 12 may be similar to, or the same as the conduit 23 described elsewhere herein.

As illustrated in FIGS. 13A and 13B, the first plunger 13 comprises a distal end 64. The distal end 64 of the first plunger 13 is configured to contact the second plunger 14. The distal end 64 of the first plunger 13 is convex, for example conical. The distal end 64 of the first plunger 13 comprises an apex 66. The distal end 64 of the first plunger 13 comprises a base 68. In the illustrated configuration, the apex 66 is at a central portion of the distal end 64 of the first plunger 13. The apex 66 is closer to the valve 39 of the second plunger 14 than the base 88. The apex 66 is closer to the dilution chamber port 51 than the base 68.

The second plunger 14 comprises a proximal end 70. The proximal end 70 of the second plunger 14 is configured to contact the first plunger 13. The proximal end 70 of the second plunger 14 is concave, for example it may have an inverse-conical profile. That is, the proximal end 70 of the second plunger 14 defines a concave, e.g. conical recess. The proximal end 70 of the second plunger 14 is configured to receive the convex, e.g. conical, distal end 64 of the first plunger 13. In other examples, the distal end 66 of the first plunger may be concave and the proximal end 70 of the second plunger may be convex, as described in FIGS. 10 and 26.

### Method 1400 for Preparing the Medication Delivery Apparatus 2

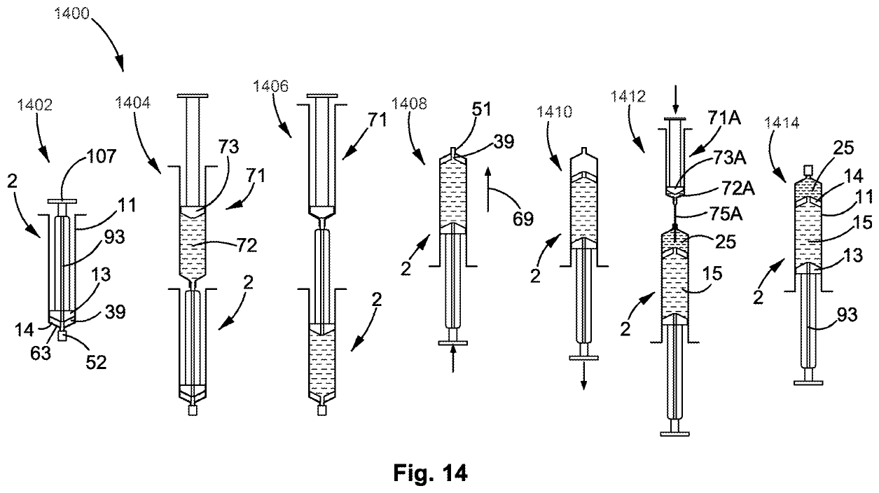

FIG. 14 illustrates a method 1400 of preparing the medication delivery apparatus 2 described with reference to FIGS. 12 to 13B, according to some embodiments. That is, the method 1400 is for preparing a medication delivery apparatus 2 that comprises a plunger lumen 93 as described herein.

At 1402, the medication delivery apparatus 2 is in an initial state. In the initial state, the first plunger 13 is in contact with the second plunger 14. Furthermore, the second plunger 14 is in contact with the distal end 63 of the container 11. In the initial state, the dilution chamber port cap 52 is connected to the medication delivery apparatus 2.

At 1404, the clinician removes the first plunger cap 107. The clinician connects a filling syringe 71 to the first plunger connector 96.

At 1406, the clinician uses the first filling syringe 71 to fill the active agent chamber 15 with the pharmaceutical preparation. The first filling syringe 71 comprises a first filling syringe chamber 72 that comprises the pharmaceutical preparation. The clinician actuates a first filling syringe plunger 73 to displace the pharmaceutical preparation from the first filling syringe chamber 72 into the active agent chamber 15 via the plunger lumen 93.

At 1408, the clinician removes the first filling syringe 71 and moves the medication delivery apparatus 2 so that the dilution chamber port 51 is facing an upwards direction 69. The clinician moves the medication delivery apparatus 2 (e.g. by tapping the medication delivery apparatus 2) to cause bubbles that may be in the pharmaceutical preparation within the active agent chamber 15 to move towards the valve 39 and the dilution chamber port 51. The clinician then moves the first plunger 13 towards the dilution chamber port 51. This displaces air bubbles that may be present in the pharmaceutical preparation out of the valve 39 and the second outlet 51.

At 1410, the clinician moves the first plunger 13 away from the second outlet 51. This also moves the second plunger 14 away from the second outlet 51, thereby creating a volume for the dilution chamber 25.

At 1412, the clinician uses a second filling syringe 71A to fill the dilution chamber 25 with the diluent. The second filling syringe 71A comprises a second filling syringe chamber 72A that comprises the diluent. The second filling syringe 71A comprises a second filling syringe conduit 75A which may be, for example, a needle. The clinician inserts the second filling syringe conduit 75A in the dilution chamber port 51.

The clinician actuates a second filling syringe plunger 73A to displace the diluent from the second filling syringe chamber 72A through the second filling syringe conduit 75A and into the dilution chamber 25. An outer diameter of the second filling syringe conduit 75A is less than an inner diameter of the dilution chamber opening 53. Thus, bubbles that are present in the diluent when displaced into the dilution chamber 25 can flow past the second filling syringe conduit 75A and out of the dilution chamber 25 during filling.

At 1414, the clinician caps the dilution chamber port 51 with the dilution chamber port cap 52. The medication delivery apparatus 2 is thereby in a storage state, and can be stored for a later infusion, or used in an infusion.

### Alternative First Plunger 13 and Second Plunger 13 Configuration

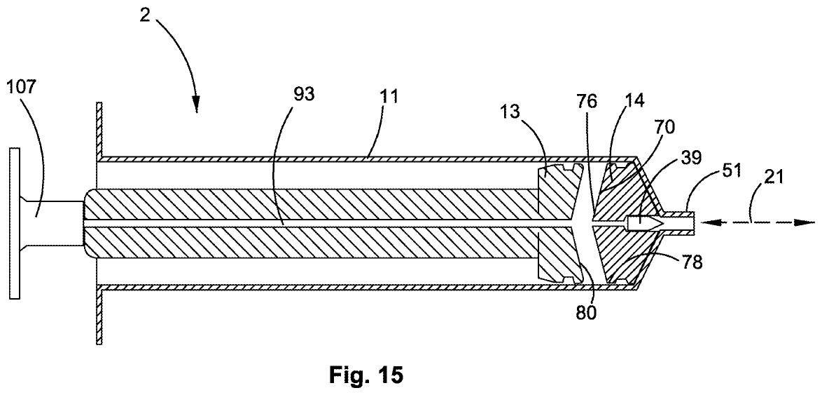

As illustrated in FIGS. 13A and 13B, the first plunger 13 comprises a conical distal end 64 and the second plunger 14 comprises a proximal end 70 with an inverted-conical profile that is configured to receive the conical distal end 64 of the first plunger 13. FIG. 15 illustrates an embodiment of the medication delivery apparatus 2 comprising an alternative first plunger 13 and an alternative second plunger 14.

The distal end 80 of the first plunger 13 of the medication delivery apparatus 2 of FIG. 15 is configured to contact the second plunger 14. In this case however, the distal end 70 of the second plunger 14 is conical. The distal end 70 of the second plunger 14 comprises an apex 76. The distal end 70 of the second plunger 14 comprises a base 78. In the illustrated configuration, the apex 76 is at a central portion of the proximal end of the second plunger 14. The apex 76 is further away from to the valve 39 of the second plunger 14 than the base 78 in a direction parallel to the longitudinal axis 21. Similarly, the apex 76 is further away from the dilution chamber port 51 than the base 78 in the direction parallel to the longitudinal axis 21.

The first plunger 13 comprises a proximal end 80. The proximal end 80 of the first plunger 13 is configured to contact the second plunger 14. The proximal end 80 of the first plunger 13 has an inverse-conical profile. That is, the proximal end 80 of the first plunger 13 defines a conical recess. The proximal end 80 of the first plunger 13 is configured to receive the conical proximal end 70 of the second plunger 14.

### Method 1600 for Preparing the Medication Delivery Apparatus 2

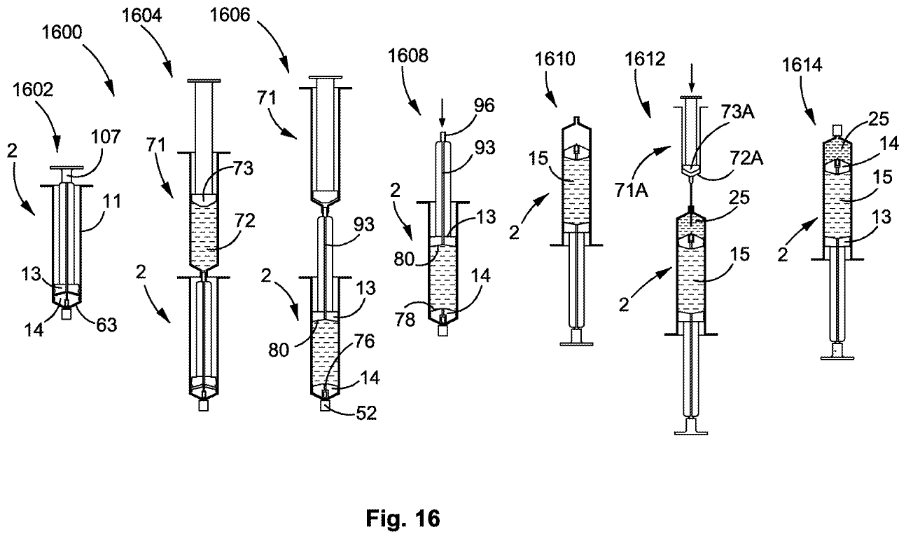

FIG. 16 illustrates a method 1600 of preparing the medication delivery apparatus 2 described with reference to FIG. 15, according to some embodiments. That is, the method 1600 is for preparing a medication delivery apparatus 2 that comprises a second plunger 14 with a proximal end 70 with a conical profile and a first plunger 13 with a distal end with an inverse-conical profile that is configured to receive the proximal end 70 of the second plunger, as described herein.

At 1602, the medication delivery apparatus 2 is in an initial state. In the initial state, the first plunger 13 is in contact with the second plunger 14. Furthermore, the second plunger 14 is in contact with the distal end 63 of the container 11. In the initial state, the dilution chamber port cap 52 is connected to the medication delivery apparatus 2.

At 1604, the clinician removes the first plunger cap 107. The clinician connects a filling syringe 71 to the first plunger connector 96.

At 1606, the clinician uses the first filling syringe 71 to fill the active agent chamber 15 with the pharmaceutical preparation. The first filling syringe 71 comprises a first filling syringe chamber 72 that comprises the pharmaceutical preparation. The clinician actuates a first filling syringe plunger 73 to displace the pharmaceutical preparation from the first filling syringe chamber 72 into the active agent chamber 15, via the plunger lumen 93.

At 1808, the clinician removes the first filling syringe 71. The clinician moves the medication delivery apparatus 2 (e.g. by tapping the medication delivery apparatus 2) to cause bubbles that may be in the pharmaceutical preparation within the active agent chamber 15 to move towards the second plunger lumen opening 97 of the first plunger 13. As the distal end 80 of the first plunger 13 has an inverse-conical profile, the air in the pharmaceutical preparation accumulates at the second plunger lumen opening 97 of the first plunger 13. The clinician then moves the first plunger 13 towards the dilution chamber port 51. This displaces air bubbles that may be present in the pharmaceutical preparation out of the plunger lumen 93 and the first plunger lumen opening 95 of the first plunger 13. Alternatively, the clinician may remove air prior to removal of the first filling syringe 71. The clinician may achieve this by actuating the first filling syringe plunger 73 to cause a vacuum pressure and aspirate the air back into the first filling syringe 71.

At 1610, the clinician reconnects the first plunger cap 107 to the first plunger connector 96. The clinician removes the dilution chamber port cap 52 from the dilution chamber port 51. The clinician moves the first plunger 13 away from the second outlet 51. This also moves the second plunger 14 away from the second outlet 51, thereby creating a volume for the dilution chamber 25.

At 1612, the clinician uses a second filling syringe 71A to fill the dilution chamber 25 with the diluent. The second filling syringe 71A comprises a second filling syringe chamber 72A that comprises the diluent. The second filling syringe 71A comprises a second filling syringe conduit 75A which may be, for example, a needle. The clinician inserts the second filling syringe conduit 75A in the dilution chamber port 51.

The clinician actuates a second filling syringe plunger 73A to displace the diluent from the second filling syringe chamber 72A through the second filling syringe conduit 75A and into the dilution chamber 25. An outer diameter of the second filling syringe conduit 75A is less than an inner diameter of the opening of the dilution chamber port 51. Thus, bubbles that are present in the diluent when displaced into the dilution chamber 25 can flow past the second filling syringe conduit 75A and out of the dilution chamber 25 during filling.

At 1614, the clinician caps the dilution chamber port 51 with the dilution chamber port cap 52. The medication delivery apparatus 2 is thereby in a storage state, and can be stored for a later infusion, or used in an infusion.

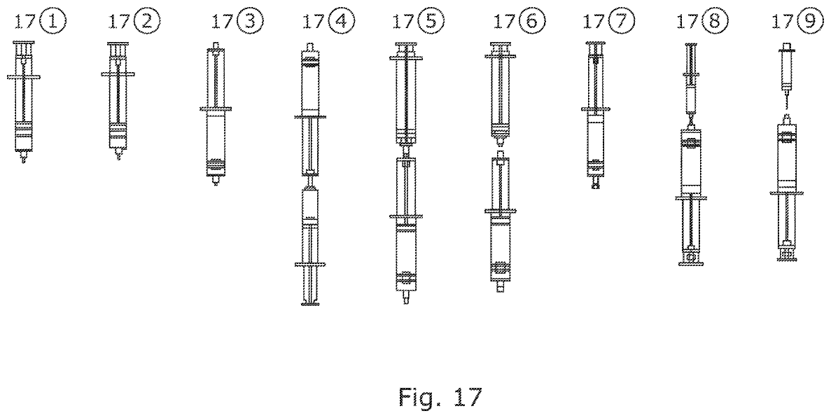

Various example methods have been described above in which the medication delivery apparatus is provided to the clinician (e.g. from factory or storage) with the active agent chamber and the dilution chamber empty and in which the clinician fills the chambers prior to use. Two further example methods of filling the active agent chamber and the dilution chamber will now be described with reference to FIGS. 17 and 18.

In both these example methods, prior to filling the dilution chamber, the second plunger 14 (also referred to as the ‘separation plunger’) is positioned at a starting position which defines an initial volume of the dilution chamber 25, which the dilution chamber is to have at a start of the infusion.

The apparatus may provided to the clinician (e.g. from the factory or storage) with the second plunger 14 at an initial position which is the same as the starting position or at an initial position which is further away from a distal end of the container than the starting position. If the initial position is not the same as the starting position, the clinician moves the second plunger to the starting position as a first part of the filling process. This movement of the second plunger prior to filling the active agent and dilution chambers, ensures that high striction of the separation plunger to the walls of the syringe that may have developed during storage can be broken and also ensures that the clinician has checked that the secondary plunger is in the correct starting position prior to use. If the initial position of the second plunger is the same as the starting position, this requires less steps to fill the device, but still helps ensure the secondary plunger is in the correct starting position prior to the infusion.

The container 11 may have a marking indicating the starting position at which the second plunger is to be positioned within the container at the start of an infusion. This assists the clinician in checking the position of the second plunger and moving the second plunger to the starting position if the second plunger is not in the starting position. The container may also have a marking showing the initial position of the second plunger (if the initial position is different to the starting position) and/or a starting position of the first plunger, which the first plunger is to have at a start of the infusion.

In some examples, the starting position is located so that the dilution chamber has a volume of 10 mL when the second plunger is in the starting position. In some examples the initial position of the second plunger is at a location which provides a dilution chamber volume of 15 mL. In this case the clinician moves the second plunger from the 15 mL mark to the 10 mL mark.

In the filling method shown in FIG. 17, the first plunger 13 is retracted and the end cap 52 removed, prior to injection of pharmaceutical preparation into the active agent chamber 15. This allows air to transit out of the open end of the syringe as the pharmaceutical preparation is injected in.

Referring to FIG. 17, at a first step 17-1 the medication delivery apparatus is received by the clinician. The apparatus has an empty dilution chamber and an empty active agent chamber. The clinician may remove the apparatus from sterile packaging at this stage. At a second step 17-2, the end cap 52 is disconnected and the first plunger 13 is pushed further into the container so as to push (through air pressure in the gap between the first and second plungers) the second plunger to the starting position. The starting position may be indicated by marking on the container. In other examples, the apparatus may be provided with the second plunger already in the starting position. At block 17-3, a plunger lumen cap (also referred to as an ‘axial fill cap’) is disconnected the first plunger is then retracted to a starting position for the first plunger. The starting position for the first plunger may for example be marked on the container 11 or may be found by retracting the first plunger until a gasket or other part of the first plunger makes contact with a lip, flange or projection of the container thereby stopping retraction of the first plunger. The first plunger may be retracted without moving the second plunger as air can flow through the plunger lumen into the active agent chamber 15 between the first and second plungers.

At step 17-4 pharmaceutical preparation is injected into the active agent chamber, for example by connecting a pharmaceutical preparation syringe containing pharmaceutical preparation to the plunger lumen and injecting the pharmaceutical preparation through the plunger lumen into the active agent chamber. In this step the apparatus is positioned (e.g. held) with the dilution chamber opening facing upwards.

At step 17-5, the end cap 52 is placed back on the dilution chamber opening. The apparatus is then turned the other way around with the dilution chamber opening facing downwards and the pharmaceutical preparation syringe is used to remove (e.g. aspirate) any bubbles formed in the active agent chamber 25.

At step 17-6, the plunger lumen cap is placed back on the plunger lumen. At step 17-7, the pharmaceutical preparation syringe may be disconnected from the dilution chamber opening.

At step 17-8, diluent is injected into the dilution chamber. For example, the end cap 25 may be removed from the dilution chamber opening and a diluent syringe used to inject diluent through the dilution chamber opening into the dilution chamber. At step 17-9, the diluent syringe may be removed from the dilution chamber opening, de-bubbling may be performed and the end cap 25 may be replaced on the dilution chamber opening.

The filling method shown in FIG. 18 is similar to the filling method of FIG. 17, but a major difference is that the end cap 52 is in place and the first plunger 13 is adjacent to the second plunger 2 prior to injection or aspiration of the pharmaceutical preparation into the active agent chamber. Aspiration by moving the plunger rod 94 away from the drug chamber (and the end cap 52 being in place over the end of the syringe) helps to ensure that positive pressure does not develop in the active agent chamber which could result in the pharmaceutical preparation being accidentally injected into the empty dilution chamber.

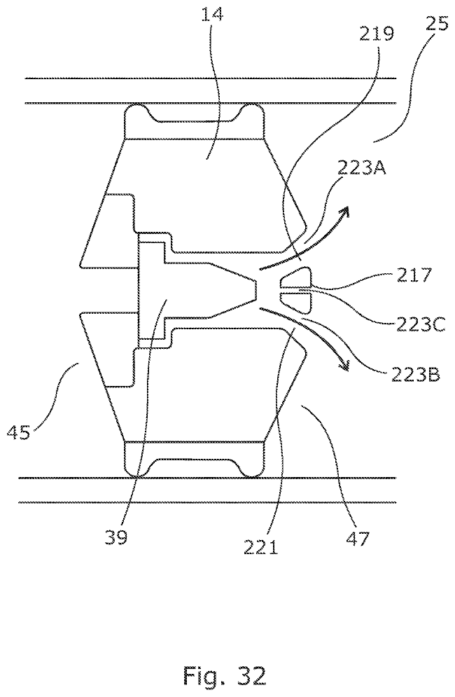

At steps 18-1 and 18-2, the end cap 25 is removed and the first plunger moved so as to move the second plunger to the starting position, similar to step 32-2 of FIG. 32. At step 18-3, the end cap 25 is replaced on the dilution chamber opening. At step 18-4, the plunger lumen cap is removed from the plunger lumen. At step 18-5, the apparatus is positioned with the dilution chamber opening pointing upwards and a pharmaceutical preparation syringe is used to inject pharmaceutical preparation into the active agent chamber via the plunger lumen, any remaining air in the active agent chamber is aspirated downwards and drawn into the pharmaceutical preparation syringe. At step 18-6, the apparatus is re-orientated so that the dilution chamber opening points downwards and air is aspirated out of the active agent chamber into the pharmaceutical preparation syringe. At step 18-7, the plunger lumen cap is replaced on the plunger lumen and the end cap 25 is removed from the dilution chamber opening. At step 18-8, the apparatus is re-orientated so that the dilution chamber opening points upwards. A diluent syringe is then connected to the dilution chamber opening. At step 18-9, the diluent syringe is used to inject diluent into the dilution chamber and air bubbles are aspirated out of the dilution chamber. At step 18-10, the end cap 25 may be replaced on the dilution chamber opening.

### Alternative Configurations of the Medication Delivery Apparatus 2

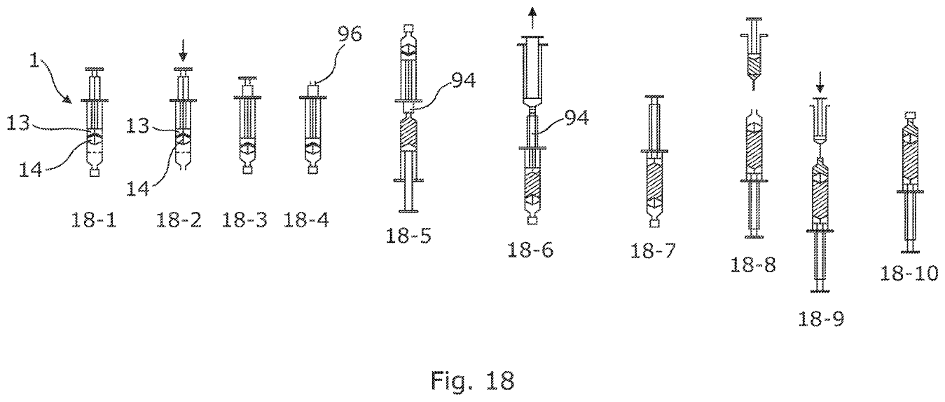

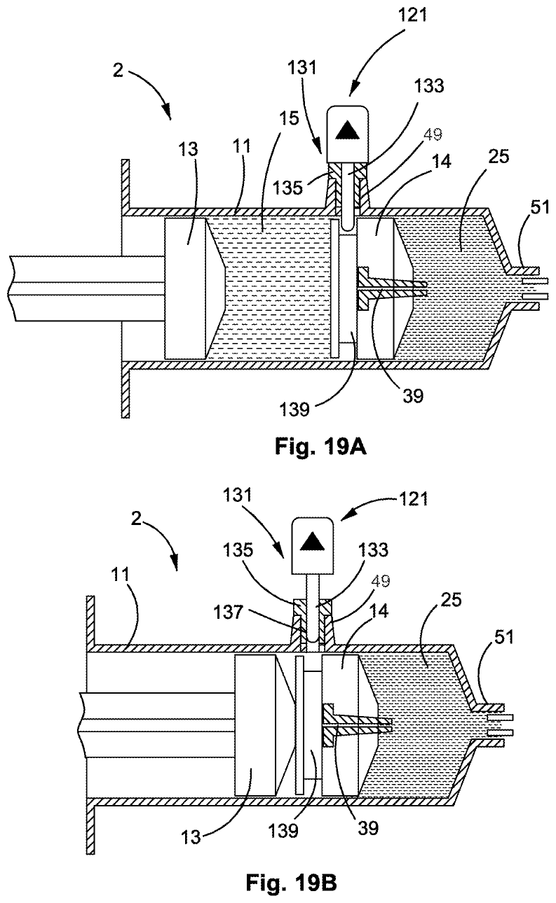

In some embodiments, one or more characteristics of the medication delivery apparatus 2 may be tailored to provide certain functional characteristics. FIGS. 19A to 24 illustrate a plurality of alternative configurations of the medication delivery apparatus 2. The embodiments of the medication delivery apparatus 2 illustrated in each of FIGS. 19 to 24 may be the same as, or similar to the medication delivery apparatus 2 described previously, in at least some respects. For example, the embodiments of the medication delivery apparatus 2 illustrated in each of FIGS. 19A to 24 comprise a first plunger 13, a second plunger 14, a container 11, a valve 39 (or valve means) and a dilution chamber port 51. One or more of at least the first plunger 13, second plunger 14, one-way valve 39 (or valve means), container 11 and the dilution chamber port 51 may be as described herein (for example, with reference to one or more of the previously described embodiments), except with respect to any differences noted in the relevant disclosure.

As previously described, the alternative configurations of the medication delivery apparatus 2 described with reference to FIGS. 19A to 24 may comprise the container 11. The container 11 is configured to receive the second plunger 14 and at least a portion of the first plunger 13. The container 11 and the second plunger 14 define a dilution chamber 25. The dilution chamber 25 is configured to receive a diluent. The second port 51 may be referred to as a dilution chamber port 51. The container 11 defines the dilution chamber opening 51. The first plunger 13, the container 11 and the second plunger 14 define an active agent chamber 15 that is configured to receive a pharmaceutical preparation. In FIG. 19A the first plunger 13 and the second plunger 14 are in a pre-infusion state in starting positions which they have at the start of the infusion. In FIG. 19B the first plunger 13 has been moved further into the container 11 so that it contacts the second plunger 14.

While FIGS. 19A to 24 show a first plunger with a convex distal surface facing the second plunger and a second plunger with a flat proximal surface facing the first plunger; the arrangement may be modified so that the first plunger has a concave distal surface and the second plunger has a convex proximal surface, for instance as described elsewhere in this application and shown in FIG. 10 or FIG. 26. Further, while the apparatus shown in FIGS. 19A to 24 has an active agent port 49 on a side of the container and active agent port cap 50, the arrangement may be modified so that there is no active agent port 49 and no active agent port cap 50. The active agent port 49, may be used for filling the active agent chamber, but when no active agent port 49 is present, the active agent chamber may be filled in other ways, for example through a proximal opening of the container 11 (e.g. if the first plunger is removed), through a plunger lumen of the first plunger, or the apparatus may be provided with the active agent chamber pre-filled.

The first plunger 13 is configured to seal with the container 11 to provide a first seal. The first seal may be as described herein. The second plunger 14 is configured to seal with the container 11 to provide a second seal. The second seal may be as described herein. The container 11 comprises an active agent chamber opening 17, as is described herein.

The second plunger 14 comprises a one-way valve 39. The valve 39 is configured to control a flow of pharmaceutical preparation from the active agent 15 chamber to the dilution chamber 25, as is described herein. The valve 39 is configured to move from a closed position to an open position upon application of a force exceeding a valve threshold force to an inlet side of the valve 39. The valve 39 is configured to move from the open position to the closed position upon removal of the force applied to the inlet side of the valve 39.

A break loose force of a plunger in a syringe may be considered a force required to break the static friction of the plunger. In the context of the medication delivery apparatus 2, a break loose force of the first plunger 13 may be considered a force required to break the static friction of the first plunger 13 (i.e. the static friction between the first plunger 13 and the container 11). That is, the break loose force of the first plunger 13 may be considered a force required to cause movement of the first plunger 13 when the first plunger 13 is stationary. The break loose force may be considered when the active agent chamber 15 comprises the pharmaceutical preparation and/or the dilution chamber 25 comprises the diluent. The break loose force of the first plunger 13 may be referred to as a first plunger break loose force. The break loose force of the first plunger 13 may be referred to as a first break loose force.

A break loose force of the second plunger 14 may be considered a force required to break the static friction of the second plunger 14 (i.e. the static friction between the second plunger 14 and the container 11). That is, the break loose force of the second plunger 14 may be considered a force required to cause movement of the second plunger 14 when the second plunger 14 is stationary. The break loose force may be considered when the active agent chamber 15 comprises the pharmaceutical preparation and/or the dilution chamber 25 comprises the diluent. The break loose force of the second plunger 14 may be referred to as a second plunger break loose force. The break loose force of the second plunger 14 may be referred to as a second break loose force.

The Valve Threshold Force May be Less than a Sum of the Break Loose Force of the Second Plunger 14 and the Break Loose Force of the First Plunger

In some embodiments, the valve threshold force is less than a sum of the break loose force of the second plunger 14 and the break loose force of the first plunger 13. In other words, the valve threshold force is less than a sum of the second break loose force and the first break loose force. Such a configuration can be beneficial in cases where the infusion device 3 is a vacuum infusion device 3. That is, such a configuration can be beneficial in cases where the infusion device 3 applies the vacuum pressure 61 to the dilution chamber port 51 of the medication delivery apparatus 2. In such cases, where the vacuum pressure 61 exerts a force on the valve 39 that is sufficient to open the valve 39, the second plunger 14 does not move.

As previously described, the infusion device 3 may apply the vacuum pressure 61 to the dilution chamber port 51 of the medication delivery apparatus 2. The vacuum pressure 61 applies a vacuum force to the fluid in the dilution chamber 25 (i.e. the diluent). As the fluid in the dilution chamber 25 is generally incompressible, the vacuum force is also applied to the second plunger 14. The vacuum force may be transmitted to the fluid in the active agent chamber 15 (i.e. the pharmaceutical preparation) via the valve 39 of the second plunger 14 if the vacuum force exceeds the valve force threshold (thereby opening the valve 39). As the vacuum force is applied to the outlet side 47 of the valve 39, the valve 39 can open and the vacuum force 61 can be transmitted to the pharmaceutical preparation in the active agent chamber 15. As the pharmaceutical preparation is a fluid, it is also generally incompressible. Thus, the vacuum force is also applied to the first plunger 13.

The vacuum force being great enough to open the valve 39 will thereby cause movement of the first plunger 13. Thus, the vacuum pressure 61 will draw the pharmaceutical preparation into the dilution chamber 25. Control of the vacuum pressure 61 thereby enables control of the flow rate of the pharmaceutical preparation into the dilution chamber 25.

The valve threshold force may be less than the break loose force of the second plunger 14. This ensures the second plunger 14 remains stationary while the pharmaceutical preparation is being drawn from the active agent chamber 15 by the vacuum pressure 61. In some embodiments, the break loose force of the second plunger 14 is greater than the break loose force of the first plunger 13.

It should be noted that, as the fluid in each of the dilution chamber 25 and the active agent chamber 15 is incompressible, for the second plunger 14 to be moved by the vacuum pressure prior to the valve 39 opening, the vacuum pressure would have to overcome both the break loose force of the first plunger 13 and the break loose force of the second plunger 14 (as both will move). As the fluid may not be completely incompressible, in some embodiments, the break loose force of the second plunger 14 should be greater than the valve threshold force.

When the valve 39 is in the open position, there is little force (at low flow rates) across the second plunger 14. This force is proportional to the resistance across the valve 39. In some embodiments, a force across the valve 39 when the valve 39 is in the open position is referred to as an open valve force. When the valve 39 is in the open position, as long as the infusion device 3 is capable of developing a vacuum pressure greater than the break loose force of the first plunger 13, the first plunger 13 will move.

When the valve 39 is in the open position, at high flow rates, the open valve force will be greater. That is, the open valve force is greater at high flow rates. In some embodiments, the break loose force of the second plunger 14 is greater than the open valve force at a maximum flow rate of the infusion. This will ensure that the second plunger 14 does not move before the first plunger 13 contacts the second plunger 14. In some embodiments, the break loose force of the second plunger is around 450 grams.

In some embodiments, the break loose force of the second plunger 14 is less than a force alarm threshold. The force alarm threshold is a force at which the infusion device 3 will alarm. The alarm of the infusion device 3 may be indicative of an occlusion. In some embodiments, the force alarm threshold may be 1,200 grams.

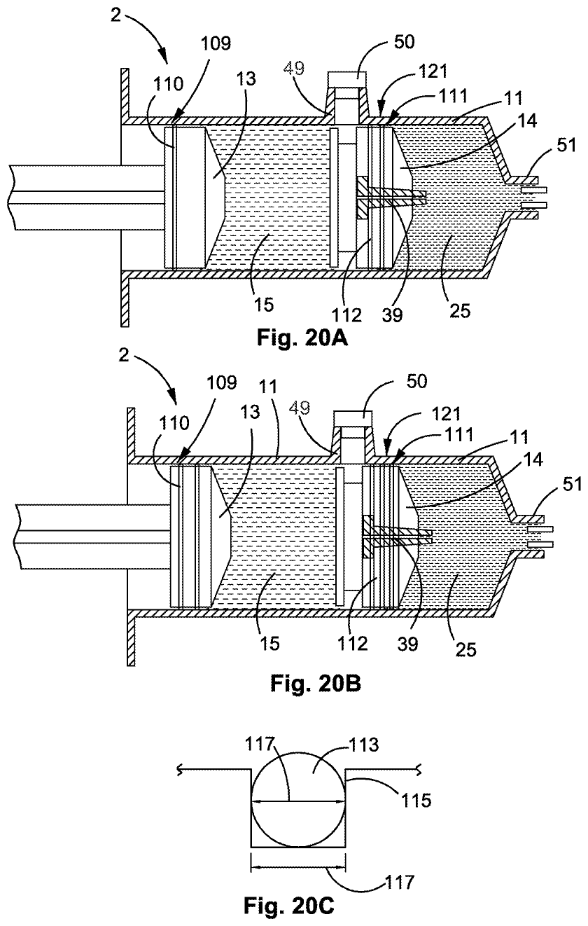

FIG. 20A illustrates a side view of a medication delivery apparatus 2 in a first state, according to some embodiments. The first state may be a pre-infusion state. The first state may be indicative of a first stage of an infusion.

FIG. 20B illustrates a side view of another medication delivery apparatus 2, in the first state, according to some embodiments. The first state may be a pre-infusion state. The first state may be indicative of a first stage of an infusion.

The medication delivery apparatus 2 illustrated in FIGS. 20A and 20B comprises a first plunger 13 and a second plunger 14. The first plunger 13 may be as described herein, in at least some embodiments. The second plunger 14 may be as described herein, in at least some embodiments. The medication delivery apparatus 2 comprises a container 11. The container 11 may be as described herein, in at least some embodiments. The medication delivery apparatus 2 comprises an active agent chamber 15. The active agent chamber 15 may be as described herein, in at least some embodiments. The medication delivery apparatus 2 comprises a dilution chamber 25. The dilution chamber 25 may be as described herein, in at least some embodiments. The medication delivery apparatus 2 comprises a active agent chamber port 49. The active agent chamber port 49 may be as described herein, in at least some embodiments. The medication delivery apparatus 2 comprises a dilution chamber port 51. The dilution chamber port 51 may be as described herein, in at least some embodiments. The medication delivery apparatus 2 comprises a valve 39. The valve 39 may be as described herein, in at least some embodiments.

As illustrated in FIGS. 20A and 20B, in some embodiments, the first plunger 13 comprises a first number of O-rings 109. The first number of O-rings 109 comprises a first plunger O-ring 110. The first number of O-rings 109 is configured to provide a number of O-ring joints between the first plunger 13 and the container 11. The number of O-ring joints corresponds to the first number of O-rings 109. That is, the number of O-ring joints is the same as the number of O-rings of the first number of O-rings 109.

Also as illustrated in FIGS. 20A and 20B, in some embodiments, the second plunger 14 comprises a second number of O-rings 111. The second number of O-rings 111 comprises a second plunger O-ring 112. The second number of O-rings 111 are configured to provide a second number of O-ring joints between the second plunger 14 and the container 11. The second number of O-ring joints corresponds to the second number of O-rings 111. That is, the second number of O ring joints is the same as the number of O-rings of the second number of O-rings 111.

In the embodiment illustrated in FIG. 20A, the first number is one. That is, the first number of O-rings 109 comprises one O-ring 110. The second number is more than one. That is, the second number of O-rings 111 comprises more than one O-ring 112, 113. In particular, the second number is two. That is, the second number of O-rings 111 comprises two O-rings 112, 113. The second number is greater than the first number. The O-rings of the first number of O-rings 109 and the second number of O-rings 111 may be the same. That is, the O-rings of the first number of O-rings 109 and the second number of O-rings 111 may have the same dimensions.

In the embodiment illustrated in FIG. 20B, the first number is two. That is, the first number of O-rings 109 comprises two O-rings. The second number is more than two. That is, the second number of O-rings 111 comprises more than two O-rings. In particular, the second number is three. That is, the second number of O-rings 111 comprises three O-rings 112, 113. The second number is greater than the first number. The O-rings of the first number of O-rings 109 and the second number of O-rings 111 may be the same. That is, the O-rings of the first number of O-rings 109 and the second number of O-rings 111 may have the same dimensions.

In the embodiments illustrated in FIGS. 20A and 20B, the O-rings are comparably sized. That is, the strength of the O-ring joint provided by each O-ring is generally similar.

FIG. 20C illustrates a cross-section of an O-ring 113, according to some embodiments. The O-ring 113 may illustrate one or more of the first number of O-rings 109 and/or the second number of O-rings 111. The O-ring 113 is positioned in an O-ring groove 115. The first plunger 13 may comprise such an O-ring groove 115. For example, one or more of the first number of O-rings 109 may be disposed in an O-ring groove like that shown in FIG. 20C. Similarly, the second plunger 14 may comprise such an O-ring groove 115. For example, one or more of the second number of O-rings 111 may be disposed in an O-ring groove like that shown in FIG. 20C.

The O-ring 113 is dimensioned with an O-ring diameter 117. The O-ring groove 115 comprises a groove width 119. The O-ring diameter 117 may be less than the groove width 119. The O-ring diameter 117 may be the same as the groove width-119. The O-ring diameter 117 may be greater than the groove width 119. In some embodiments, the diameter of one of the first number of O-rings 109 is referred to as a first diameter. Similarly, the diameter of one of the second number of O-rings 111 is referred to as a second diameter. In some embodiments, the groove within which one of the first number of O-rings 109 is disposed is referred to as a first groove. The first groove may have a first groove width. Similarly, the groove within which one of the second number of O-rings 111 is disposed is referred to as a second groove. The second groove may have a second groove width.

In some embodiments, the second diameter is greater than the first diameter. That is, the diameter of one or more of the second number of O-rings 111 is greater than the diameter of one or more of the first number of O-rings 109. This increases the break loose force associated with the O-ring joint of the relevant one of the second number of O-rings 111 when compared to the break loose force associated with the O-ring joint of the relevant one of the first number of O-rings 109.

Thus, providing the medication delivery apparatus 2 such that the valve threshold force is less than a sum of the break loose force of the first plunger 13 and the break loose force of the second plunger 14 may be accomplished in a number of ways. Similarly, providing the medication delivery apparatus 2 such that the break loose force of the second plunger 14 is greater than the break loose force of the first plunger 13 may be accomplished in a number of ways by tailoring the characteristics of the first number of O-rings 109 and the second number of O-rings 111. Similarly, providing the medication delivery apparatus 2 such that the open valve force is less than the break loose force of the second plunger 14 may be accomplished in a number of ways.

In some embodiments, the break loose force of each of the first plunger 13 and the second plunger 14 can be controlled by controlling the first number or the second number. In such cases, the dimensions associated with each O-ring may be comparable. That is, the diameter(s) of the O-ring(s) of the first number of O-rings 109 may be similar to, or the same as the diameter(s) of the O-ring(s) of the second number of O-rings 111. Thus, in cases where the second number is greater than the first number, the break loose force of the second plunger 14 is greater than the break loose force of the first plunger 13. The first number can be selected such that the valve threshold force is greater than a break loose force of the first plunger 13.

In some embodiments, the break loose force of each of the first plunger 13 and the second plunger 14 can be controlled by controlling the dimensions associated with the O-rings of the first number of O-rings 109 and the second number of O-rings 111. For example, the groove width of the grooves of the second number of O-rings 111 can be sized to be less than the groove width of the grooves of the first number of O-rings 109. In such cases, the O-ring joints provided by the second number of O-rings 111 will require more force to be overcome, thereby increasing the break loose force of the second plunger 14. In such cases, these dimensions can be controlled such that the break loose force of the second plunger 14 is greater than the break loose force of the first plunger 13. Similarly, these dimensions can be controlled such that the valve threshold force is less than a sum of the break loose force of the first plunger 13 and the break loose force of the second plunger 14. Similarly, these dimensions can be controlled such that the valve threshold force is less than the break loose force of the second plunger 14. Similarly, these dimensions can be controlled such that the open valve force is less than the break loose force of the second plunger 14.

In some embodiments, the at least one resistance element 121 comprises one or more of the second number of O-rings 111. In some embodiments, each of the second number of O-rings 111 is considered to be one of the at least one resistance element 121.

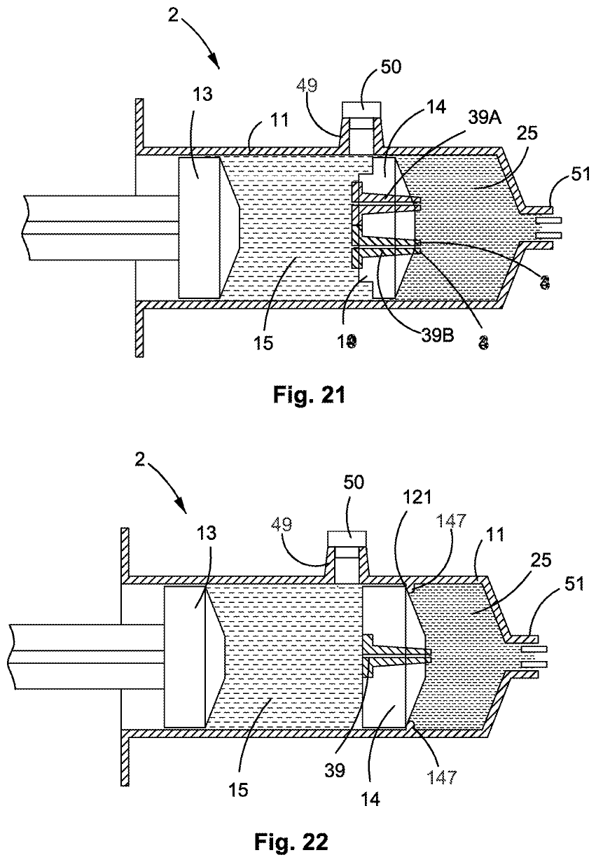

FIG. 21 illustrates a side view of another medication delivery apparatus 2, in the first state, according to some embodiments. The first state may be a pre-infusion state. The first state may be indicative of a first stage of an infusion.

The medication delivery apparatus 2 illustrated in FIG. 21 comprises a first plunger 13 and a second plunger 14. The first plunger 13 may be as described herein, in at least some embodiments. The second plunger 14 may be as described herein, in at least some embodiments. The medication delivery apparatus 2 comprises a container 11. The container 11 may be as described herein, in at least some embodiments. The medication delivery apparatus 2 comprises an active agent chamber 15. The active agent chamber 15 may be as described herein, in at least some embodiments. The medication delivery apparatus 2 comprises a dilution chamber 25. The dilution chamber 25 may be as described herein, in at least some embodiments. The medication delivery apparatus 2 comprises a active agent chamber port 49. The active agent chamber port 49 may be as described herein, in at least some embodiments. The medication delivery apparatus 2 comprises a dilution chamber port 51. The dilution chamber port 51 may be as described herein, in at least some embodiments.

As described herein, the second plunger 14 comprises the valve 39. In some embodiments, the second plunger 14 comprises a valve arrangement 149. The valve arrangement 149 is configured to control the flow of pharmaceutical preparation from the active agent chamber 15 to the dilution chamber 25. The valve arrangement 149 may comprise the valve 39. As described herein, the valve 39 may be a duckbill valve 41. The valve 39 may comprise a plurality of flaps 43.

In some embodiments, the valve arrangement 149 comprises a plurality of valves 39. One or more of the plurality of valves may be a duckbill valve 41. One or more of the plurality of valves 39 may comprise a plurality of flaps 43. For example, the valve arrangement 149 may comprise a first valve 39A and a second valve 39B. Each of the first valve 39A and the second valve 39B are configured to control the flow of pharmaceutical preparation from the active agent chamber 15 to the dilution chamber 25.

Providing the medication delivery apparatus 2 such that the valve threshold force is less than a sum of the break loose force of the first plunger 13 and the break loose force of the second plunger 14 may be accomplished in a number of ways. Providing the medication delivery apparatus 2 such that the valve threshold force is less than the break loose force of the second plunger 14 may be accomplished in a number of ways. Similarly, providing the medication delivery apparatus 2 such that the break loose force of the second plunger 14 is greater than the break loose force of the first plunger 13 may be accomplished in a number of ways. Similarly, providing the medication delivery apparatus 2 such that the open valve force is less than the break loose force of the second plunger 14 may be accomplished in a number of ways.

In some embodiments, the break loose force of the second plunger 14 can be controlled by controlling an aspect of the valve 39 and/or the valve arrangement 149. Similarly, the open valve force can be controlled by controlling an aspect of the valve 39 and/or the valve arrangement 149. For example, a material from which the valve 39 is manufactured can influence the force required to open the valve 39, and also the open valve force (i.e. the force across the valve 39 when the valve 39 is in the open position). Similarly, the dimensions of the flaps 43 can influence the force required to open the valve 39, and also the open valve force. The number of valves of the valve arrangement 149 can also influence the force required to open the valve 39 and also the open valve force. As the open valve force is proportional to the resistance of each valve 39 of the valve arrangement 149, adding more valves 39 or changing the resistance of the valves 39 (e.g. by changing a cross-sectional area of the respective valve 39 when open by modifying the valve material(s), geometry, durometer, flap configuration, size of opening etc.) can decrease the resistance across the valve arrangement 149, thereby decreasing the open valve force.

The valve threshold force, the break loose force of the first plunger 13 and the break loose force of the second plunger 14 may be controlled as described herein to ensure that the valve threshold force is less than a sum of the break loose force of the first plunger 13 and the break loose force of the second plunger 14 and/or is less than the break loose force of the second plunger 14. The valve arrangement 149 can also be controlled such that the valve threshold force is less than the break loose force of the second plunger 14. For example, the material(s) from which the valve(s) 39 of the valve arrangement 149 are manufactured can be modified to reduce the valve threshold force. Similarly, the open valve force and the break loose force of the second plunger 14 may be controlled as described herein to ensure that the open valve force is less than the break loose force of the second plunger 14. For example, the open valve force can be decreased by increasing the number of valves 39 in the valve arrangement 149 or increasing the size of an opening of a valve 39 of the valve arrangement 149 when that valve 39 is open.

FIG. 22 illustrates a side view of a medication delivery apparatus 2 in a first state, according to some embodiments. The first state may be a pre-infusion state. The first state may be indicative of a first stage of an infusion.

The medication delivery apparatus 2 illustrated in FIG. 22 comprises a first plunger 13 and a second plunger 14. The first plunger 13 may be as described herein, in at least some embodiments. The second plunger 14 may be as described herein, in at least some embodiments. The medication delivery apparatus 2 comprises a container 11. The container 11 may be as described herein, in at least some embodiments. The medication delivery apparatus 2 comprises an active agent chamber 15. The active agent chamber 15 may be as described herein, in at least some embodiments. The medication delivery apparatus 2 comprises a dilution chamber 25. The dilution chamber 25 may be as described herein, in at least some embodiments. The medication delivery apparatus 2 comprises a active agent chamber port 49. The active agent chamber port 49 may be as described herein, in at least some embodiments. The medication delivery apparatus 2 comprises a dilution chamber port 51. The dilution chamber port 51 may be as described herein, in at least some embodiments. The medication delivery apparatus 2 comprises a valve 39. The valve 39 may be as described herein, in at least some embodiments.

In the embodiment shown in FIG. 22, the medication delivery apparatus 2 comprises a projection 147. The projection 147 is configured to inhibit displacement of the second plunger 14. In particular, the projection 147 is configured to inhibit displacement of the second plunger 14 towards the dilution chamber port 51. The container 11 comprises the projection 147. The projection 147 projects inwardly. The projection 147 is an annular projection. The projection 147 extends around an interior of the container 11. The projection 147 is between the active agent chamber port 49 and the dilution chamber port 51. The projection 147 can be used to increase the break loose force of the second plunger 14.

Providing the medication delivery apparatus 2 such that the valve threshold force is less than a sum of the break loose force of the first plunger 13 and the break loose force of the second plunger 14 may be accomplished in a number of ways. Similarly, providing the medication delivery apparatus 2 such that the break loose force of the second plunger 14 is greater than the break loose force of the first plunger 13 may be accomplished in a number of ways. Similarly, providing the medication delivery apparatus 2 such that the open valve force is less than the break loose force of the second plunger 14 may be accomplished in a number of ways. In some embodiments, one or more of these is accomplished at least in part by appropriately dimensioning the projection 147.

In some embodiments, the break loose force of the second plunger 14 can be controlled by controlling an aspect of the projection. For example, the extent to which the projection 147 projects into the container 11 can be controlled. The extent to which the projection 147 extends into the container 11 from the inner surface 55 of the container 11 may be a projection dimension. This may also be referred to as a radial projection dimension. A thickness of the projection 147 in the direction parallel to the longitudinal axis 21 may be a longitudinal projection dimension. The projection dimension and the longitudinal projection dimension may also be controlled to control the break loose force of the second plunger 14.

Increasing the extent to which the projection 147 projects into the container 11 can increase the break loose force of the second plunger 14, as one has to overcome a resistive force applied to the second plunger 14 by the projection 147. Similarly, increasing the longitudinal projection dimension can increase the break loose force of the second plunger 14. Therefore, the break loose force of the second plunger 14 can be greater than the break loose force of the first plunger 13, at least in part as a result of the resistance to the movement of the second plunger 14 provided by the projection 147. The dimensions of the projection 147 (e.g. the projection dimension) may be controlled as described herein to ensure that the valve threshold force is less than a sum of the break loose force of the first plunger 13 and the break loose force of the second plunger 14. Similarly, the dimensions of the projection 147 may be controlled as described herein to ensure that the valve threshold force is less than the break loose force of the second plunger 14.

In some embodiments, the resistance element 121 comprises the projection 147.

The break loose force of each of the first plunger 13 and the second plunger 14 may be controlled by controlling the number of O-rings associated with each plunger and the dimensions of the O-rings as described above. However, other ways of controlling the break loose forces are possible. In some examples, the break loose force may be controlled through the number of valves as shown in FIG. 21 or by a providing a projection 147 which inhibits movement of the second plunger as shown in FIG. 22. In still other examples, there may be a stopping system 131 comprising an actuation element 133 which is movable into an engaging position in which it engages with a groove 139 of the second plunger to inhibit movement of the second plunger (as shown in FIG. 19A) and to a non-engaging position (shown in FIG. 19B) in which the actuation element 133 does not engage with the groove 139 of the second plunger and does not inhibit movement of the second plunger. In still other examples (not shown), a first portion of the container inner surface near the distal end of the container and starting position of the second plunger may have a rougher surface than a second portion of the container inner surface towards the proximal end of the container, so that the rougher first portion of the surface inhibits movement of the second plunger past the starting position towards the distal end of the container. In still other examples, the break loose force may be controlled by a spring between the distal end of the container and the distal surface of the second plunger or by providing an engagement member on the proximal surface of the second plunger to inhibit movement of the second plunger.

### The Second Plunger 14

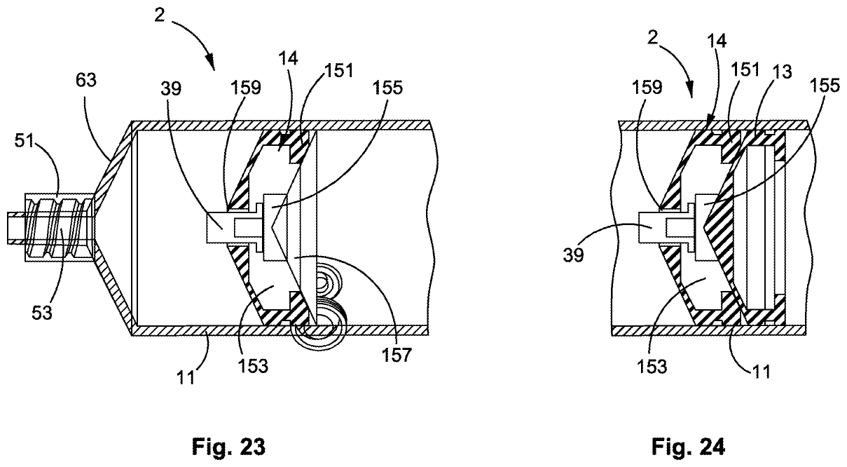

FIG. 24 illustrates a side view of a partial cross-section of a medication delivery apparatus 2, according to some embodiments. An embodiment of the second plunger 14 is illustrated in FIG. 23. The second plunger 14 of FIG. 23 comprises a second plunger sealing portion 151. The second plunger 14 comprises a second plunger sealing support portion 153. The second plunger sealing support portion 153 is configured to connect to the second plunger sealing portion 151. The second plunger sealing support portion 153 is configured to support the second plunger sealing portion 151. In some embodiments, the second plunger sealing portion 151 comprises an elastomeric material. In some embodiments, the sealing support portion 153 comprises a non-elastomeric or relatively rigid material (more rigid than the material of the sealing portion 151). The second plunger sealing support portion 153 assists the second plunger sealing portion 151 in maintaining its shape. The second plunger sealing support portion 153 may connect to the first plunger sealing portion 151 with a soft-push connection. That is, the second plunger sealing support portion 153 may be received by a recess in the second plunger sealing portion 151. The second plunger comprises a valve 39. The valve 39 may be as described herein. The second plunger 14 comprises a valve connector portion 155. The valve connector portion 155 is configured to securely connect the valve 39 to the second plunger sealing support portion 153.

FIG. 23 illustrates a side view of a partial cross-section of a medication delivery apparatus 2, according to some embodiments. The second plunger 14 comprises a second plunger recess 157. The second plunger recess 157 is a conical recess. The second plunger recess 157 is configured to receive the first plunger 13.

The valve 39 extends beyond an apex 159 of the second plunger 14 illustrated in FIGS. 23 and 24. An outer dimension of the valve 39 may be greater than an outer dimension of a channel of the dilution chamber port 51. The valve 39 may therefore contact an inner surface of the container 11 when the second plunger 14 is moved towards the distal end 63 of the container. The valve 39 may therefore prevent or minimise the extent to which the second plunger contacts the distal end 63 of the container 11. This can reduce the extent to which the second plunger 14 adheres to the distal end 63 of the container 11, thereby improving the ease with which the second plunger 14 can be removed from the distal end 63 of the container 11 to, for example, be reset for a second infusion.

Furthermore, the described valve 39 design improves the ease with which the medication delivery apparatus 2 can be primed. If the second plunger 14 were to be shaped to correspond with the shape of the distal end 63 of the container 11, the second plunger 14 may be suctioned to the distal end 63 of the container 11 before priming, which could increase the difficulty of filling the dilution chamber 25. Providing a second plunger 14 as described may overcome this problem.

In some embodiments, the valve 39 may comprise a projection (not shown) extending in a generally forward direction (i.e. away from the valve 39 and towards the dilution chamber port 51). This projection may act to contact the container 11 to minimise the extent to which the second plunger 14 suctions to the container 11. In some embodiments, rather than the valve 39 extending beyond the apex 159 of the second plunger 14, the valve 39 may be contained within the second plunger 14.

In some embodiments, the valve 39 is sized so that it does not contact the container 11. Specifically, the valve 39 does not contact the dilution chamber port 51. This reduces distortion of the valve 39 that may occur due to contact.

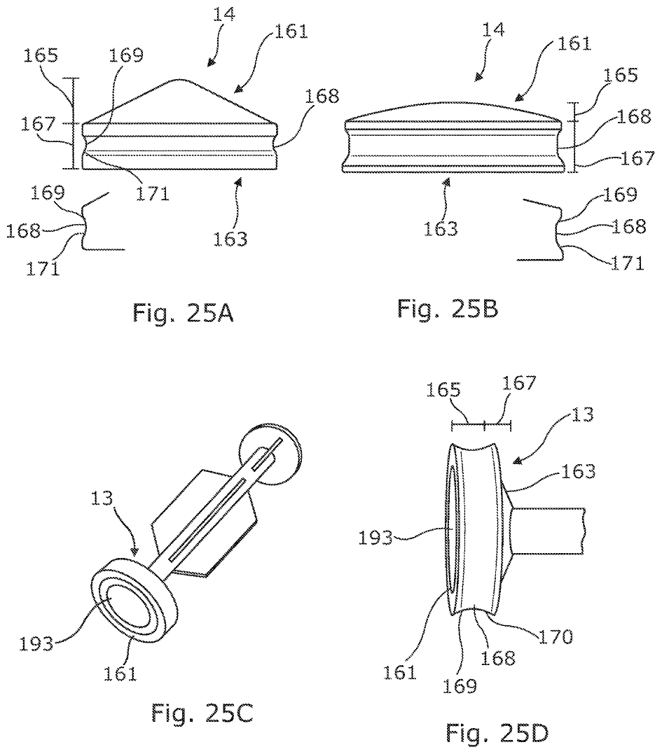

FIG. 25A illustrates a second plunger 14, according to some embodiments. The second plunger 14 of FIG. 25A comprises a front portion 161. The second plunger 14 of FIG. 27A comprises a rear portion 163. The front portion 161 is conical. The front portion 161 extends away from the rear portion 163 by a front portion depth 165. The rear portion 163 extends away from the front portion 161 by a rear portion depth 167. The front portion depth 165 is greater than the rear portion depth 167. In some embodiments, the front portion depth 165 is less than the rear portion depth 167.

The rear portion 163 comprises a rear portion groove 168. The rear portion groove 168 comprises a first angled edge 169 and a second angled edge 171. An angle between the second angled edge 171 and a longitudinal axis 173 of the second plunger 14 is greater than an angle between the first angled edge 169 and the longitudinal axis 173 of the second plunger 14.

FIG. 25B illustrates a second plunger 14, according to some embodiments. The second plunger 14 of FIG. 25B comprises a front portion 161. The second plunger 14 of FIG. 25B comprises a rear portion 163. In some embodiments, the front portion 161 is conical. In some embodiments, the front portion 161 is semi-spherical. The front portion 161 extends away from the rear portion 163 by a front portion depth 165. The rear portion 163 extends away from the front portion 161 by a rear portion depth 167. The front portion depth 165 is less than the rear portion depth 167. In some embodiments, the front portion depth 165 is greater than the rear portion depth 167.

The rear portion 163 comprises a rear portion groove 168. The rear portion groove 168 comprises a first angled edge 169 and a second angled edge 171. In some embodiments, an angle between the second angled edge 171 and a longitudinal axis 173 of the second plunger 14 is the same as an angle between the first angled edge 169 and the longitudinal axis 173 of the second plunger 14. In some embodiments, an angle between the second angled edge 171 and a longitudinal axis 173 of the second plunger 14 is greater than an angle between the first angled edge 169 and the longitudinal axis 173 of the second plunger 14. In some embodiments, an angle between the second angled edge 171 and a longitudinal axis 173 of the second plunger 14 is less than an angle between the first angled edge 169 and the longitudinal axis 173 of the second plunger 14.

Various features may be provided to inhibit or prevent the second plunger 14 from flipping or rotating out of alignment within the container 11. In some embodiments, the second plunger has a length along a longitudinal axis of the container which is at least 9 mm. This helps to prevent the second plunger from flipping out of alignment. This length of at least 9 mm may, for example, be a length between a distal side of the second plunger facing the dilution chamber opening and a proximal side of the second plunger facing the first plunger. In some examples, the length 167 of a part of the second plunger in contact with the container walls may be at least 9 mm. In some examples the length is 9 mm-11 mm.

In some examples, the rear portion depth 167 is greater than the front portion depth 165, which may help to keep the second plunger peripheral edges in contact with the internal wall of the container 11 and prevent the second plunger 14 from flipping within the container.

In some embodiments, the inner surface 55 of the container 11 may be lubricated. For example, the inner surface 55 of the container 11 may be lubricated with an oil. This lubrication can reduce the likelihood that the second plunger 14 will flip or rotate out of alignment. This may be achieved by improving the extent to which the second plunger 14 can slide. In some embodiments, a height of the second plunger 14 (i.e. a dimension of the second plunger 14 in a direction parallel to the longitudinal axis 21g) can be increased to reduce the likelihood that the second plunger 14 will flip or rotate out of alignment. In some embodiments, the size of the second plunger 14 is controlled such that the compression of the second plunger 14 is generally constant around its perimeter. For example, the second plunger 14 may be sized such that the lateral compression at a leading edge of the second plunger 14 is generally the same as the lateral compression at a trailing edge of the second plunger 14. This may help to prevent the second plunger from flipping out of alignment.

In some embodiments, the distal end 63 of the container 11 may be considered to define a concave, e.g. conical recess. The second plunger 14 is described to be moved to contact the container 11 as is described herein. In some embodiments, the distal end (e.g. first portion 161) of the second plunger 14 may be convex, e.g. conical, as is described herein. The height of the second plunger 14 may, however, be such that the convex distal surface of the second plunger 14 (i.e. the curved surface of the front portion 161 of the second plunger 14) is not directly aligned with, does not conform to, or only partially conforms to, an inner surface of the distal end 63 of the container 11. For example, the convex (e.g. conical) shape of the distal end of the second plunger 14 and the concave (e.g. conical) recess of the distal end 63 of the container 11 may have different profiles. This can advantageously reduce the extent to which the second plunger 14 may suction to the container 11. This configuration may also advantageously enables the curved surface of the front portion 161 of the second plunger 14 to seal against the dilution chamber port 51. As a result, fluid leaving the valve 39 can be directed through the dilution chamber port 51 without entering the dilution chamber 25. This may be useful when filling the medication delivery apparatus 2.

FIGS. 25C and 25D illustrate a first plunger 13, according to some embodiments. The first plunger 13 of FIGS. 25C and 25D comprises a front portion 161. The first plunger 13 of FIGS. 25C and 25D comprises a rear portion 163. The front portion 161 of the first plunger 13 is configured to contact the second plunger 14. The front portion 161 has a concave (e.g. inverse-conical) profile. That is, the front portion 161 of the first plunger 13 defines a concave (e.g. conical) recess 193. The concave (e.g. conical) recess 193 may have a profile corresponding to a truncated cone.

The front portion 161 extends away from the rear portion 163 by a front portion depth 165. The rear portion 163 extends away from the front portion 161 by a rear portion depth 167. The front portion depth 165 is greater than the rear portion depth 167. In some embodiments, the front portion depth 165 is less than the rear portion depth 167.

The first plunger 13 comprises a first plunger groove 168. The first plunger groove 168 comprises a first angled edge 169 and a second angled edge 171. In some embodiments, an angle between the second angled edge 171 and a longitudinal axis of the first plunger 13 is the same as an angle between the first angled edge 169 and the longitudinal axis of the first plunger 143 In some embodiments, an angle between the second angled edge 171 and a longitudinal axis of the first plunger 13 is greater than an angle between the first angled edge 169 and the longitudinal axis of the first plunger 13. In some embodiments, an angle between the second angled edge 171 and a longitudinal axis of the first plunger 13 is less than an angle between the first angled edge 169 and the longitudinal axis of the first plunger 13.

FIG. 26 illustrates an embodiment of the medication delivery apparatus 2, according to some embodiments

In the embodiment of FIG. 26, the first plunger 13 has a concave (distal) surface 195 facing the second plunger 14. For example the distal face of the first plunger 13 may define a concave recess. The concave recess may have a conical profile. The second plunger 14 has a convex (distal) surface 197 facing the first plunger. For example, the proximal face of the second plunger may comprise a frustoconical portion 197. While not shown in FIG. 26, the second plunger may include a valve, as previously described in the other embodiments.

When the first plunger 13 contacts the second plunger 14, an air gap 199 is defined between the first plunger 13 and the second plunger 14. The air gap 199 enables air to accumulate within the medication delivery apparatus 2, preventing or minimising the extent to which the air is injected into the dilution chamber 25 and/or the patient. The air gap may thus act as a bubble trap. In some embodiments, the air gap 199 may be defined by a concave (e.g. conical) recess 195 of the first plunger 13 and a frustoconical portion 197 of the second plunger 14. While the air gap 199 in FIG. 26 is formed between a central portion of the plungers, in other examples the air gap could have a peripheral positon off to one side.

In general terms, first plunger and second plunger may be shaped such that when the first plunger is moved into contact with the second plunger there is an air gap between the first plunger and the second plunger. The second plunger may partially conform to the shape of the first plunger so as to minimise wastage of pharmaceutical preparation, but as there is an air gap this allows space for air bubbles to be trapped between the first plunger and the second plunger when the first plunger is moved into contact with the second plunger.

The second plunger 14 may have a (distal) convex surface 198 facing the distal end 11A of the container 11. The distal end 11A of the container is the end of the container which defines the dilution chamber opening 51. The second plunger 14 is movable towards the distal end 11A of the container up to an end position at which the second plunger cannot be moved further towards the distal end of the container. For example, the end positon of the second plunger may be a position in which the second plunger 14 abuts against the distal end 11A of the container. The distal side 198 of the second plunger 14 is shaped such that in the end position there is a gap between at least part of the second plunger and the distal end of the container. The gap helps to prevent the second plunger from adhering to the distal end of the container (due to suction or otherwise). For example, the distal face of the second plunger may have a different shape or profile to a distal end of the container so as to prevent suction or adhering of the second plunger to the distal end of the container.

The distal side or face 198 of the second plunger may partially conform to the distal end of the container 11A so as to minimise wastage of pharmaceutical preparation and/or diluent when the second plunger 14 is moved into contact with the distal end of the container so as to expel the contents of the dilution chamber 25 through the dilution chamber opening 51. When the second plunger 14 is moved into contact with the distal end 11A of the container, there will still be a gap between at least part of the second plunger and the distal end of the container due to the second plunger only partially conforming to the distal end of the container. This gap helps to prevent a suction force adhering the second plunger to the distal end of the container. This may be achieved, for instance, by the distal end of the second plunger and the distal end of the container having different profiles.

In order to achieve this gap, the distal side of the second plunger facing the distal end of the container may include a first part which abuts against the distal end of the container in the end position and a second part which does not abut against the distal end of the container in the end position. For example, the first part may be a part which projects from the second plunger, the valve (if the valve protrudes from the second plunger) or a part of the distal surface of the second plunger. In some examples, the first part is a part of the distal surface of the second plunger, and 5%-50% of the distal surface area of the second plunger contacts or abuts the distal end of the container when the second plunger is moved into contact with the distal end of the container.

As discussed previously, the second plunger has a one-way valve. In some examples, as shown in FIG. 27, the one-way valve 39 may be contained inside a main body 14M of the second plunger 14. This helps to protect the one-way valve from being distorted by contact with edges of the container 11. In some examples, the main body 14M of the second plunger 14 includes at least one outlet opening 223 and at least one internal channel 222 leading from an outlet of the one-way valve 39 to the at least one outlet opening 223. In some examples, the one-way valve 39 may be a duck-bill valve.

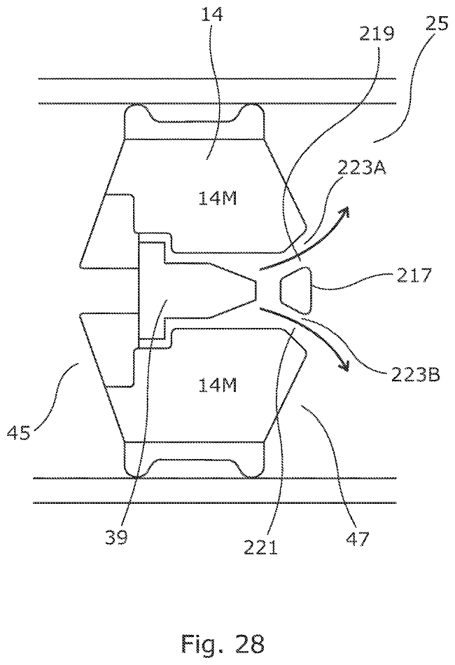

In FIG. 27 there is one outlet opening 223. In other examples the main body of the second plunger 14 may have two or more outlet openings. FIG. 28 shows a similar arrangement to FIG. 27 in which like reference numerals denote like parts, but in which the main body 14M has two outlet openings 223A and 223B. A first internal channel 219 of the main body leads to the first outlet opening 223A and a second internal channel 221 leads to the second outlet opening 223B. In this case reference numeral 217 denotes a part of the main body separating the first and second channels.

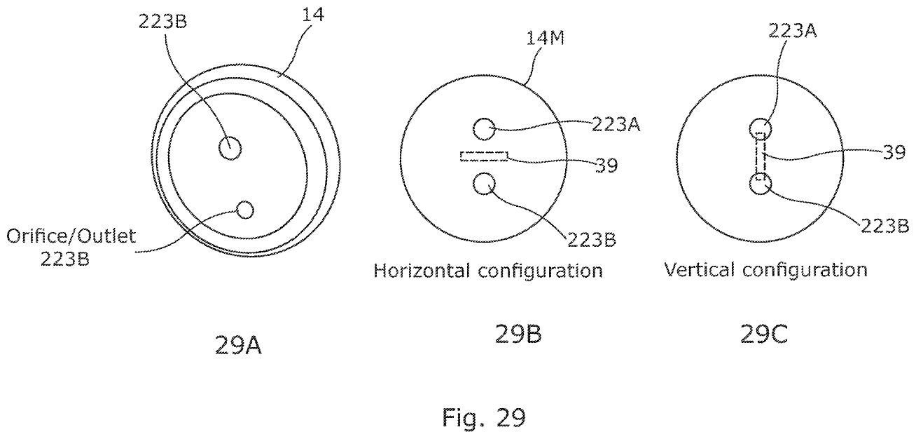

FIG. 29A is a perspective view from the front (distal end) of the second plunger 14 of FIG. 28, showing the first and second outlet openings 223A, 223B. FIG. 29B is a plan view showing one possible arrangement in which the one-way valve is arranged horizontally relative to the outlet openings. That is the one-way valve, which may be a duck-bill valve, has a slit which is substantially perpendicular to a line joining a first opening 223A and the second opening 223B. In other examples, the one-way valve may have a different orientation. For example, as shown in FIG. 29C, the valve may have a vertical orientation with the valve slit parallel with a line joining the first outlet opening 223A and the second outlet opening 223B.

In some examples, there may be a dispersion member disposed on the outlet side 47 of the valve 39. The dispersion member may be disposed in a flow path of fluid that flows through the valve 39. The dispersion member may be configured to disperse the fluid that flows through the valve 39. This can improve mixing of the fluid in the dilution chamber 25. In some examples, the dispersion member may define a first dispersion channel 219 and a second dispersion channel 221. The fluid flowing through the valve 39 is forced through the first dispersion channel 219 and the second dispersion channel 221, increasing the extent to which it mixes within the dilution chamber 25.

In some embodiments, the at least two outlet openings 223A, 223B and/or the internal channels (or dispersion channels) are configured to generate a first jet of pharmaceutical preparation directed toward a first corner of the dilution chamber 25 and a second jet of pharmaceutical preparation directed toward a second corner of the dilution chamber 25 when pharmaceutical preparation is forced from the active agent chamber through the one-way valve. In some embodiments, the apparatus is configured such that the first and second jets of pharmaceutical preparation rebound from an internal surface of the dilution chamber thereby promoting retrograde mixing of the pharmaceutical preparation with diluent in the dilution chamber.

In some implementations, the outlet opening and channels may be formed by a dispersion member which extends into a central portion of the flow path of fluid flowing through the valve 39 from an outer portion of the flow path. The dispersion member may defines a dispersion channel. The direction of fluid flowing through the dispersion channel is changed by the dispersion member. This increases the extent to which the fluid mixes within the dilution chamber 25.

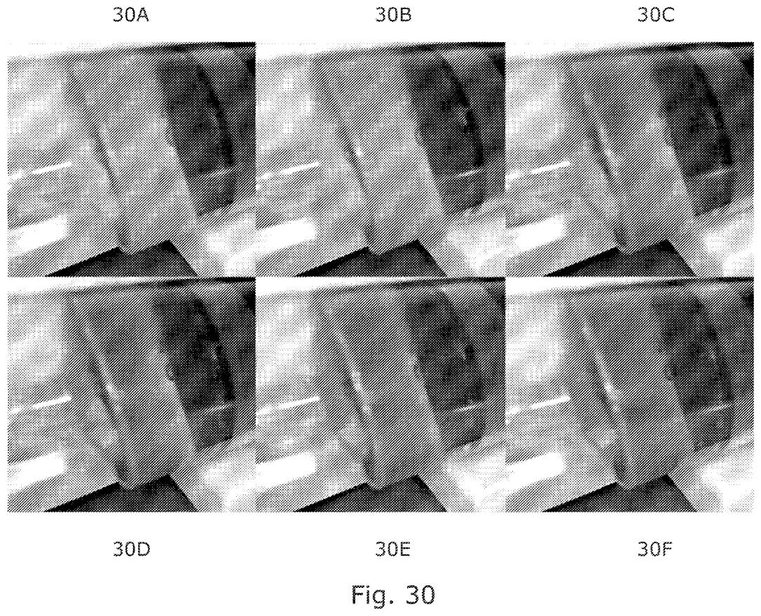

An example of the jets and mixing are illustrated in FIGS. 30A to 30F. FIGS. 30A to 30F show a priming process, but similar jets and mixing will occur during an infusion. FIG. 30A shows a first time, for instance 0.25 seconds after commencing priming, at which two jets are generated. FIG. 30B shows a second time, for instance after 0.41 seconds, where each of the two jets has reached a respective corner of the dilution chamber. FIG. 30C shows a third time, for instance after 1.51 seconds, in which the two jets have reached the dilution chamber outlet. FIG. 30D shows a fourth time, for instance after 1.95 seconds, at which retrograde mixing starts to occur. FIG. 30E shows a fifth time, for example, after 2.15 seconds at which the pharmaceutical preparation starts to enter a conduit (e.g. extension tubing) attached to the dilution chamber. FIG. 30F shows a sixth time, for example after 2.90 seconds at which good mixing has occurred.

In some embodiments, the main body of the second plunger may comprise three outlet openings. In this case the middle opening of the three outlet openings, and/or an internal channel leading to the third outlet opening, may be configured to generate a third jet of pharmaceutical preparation directed toward the dilution chamber opening.

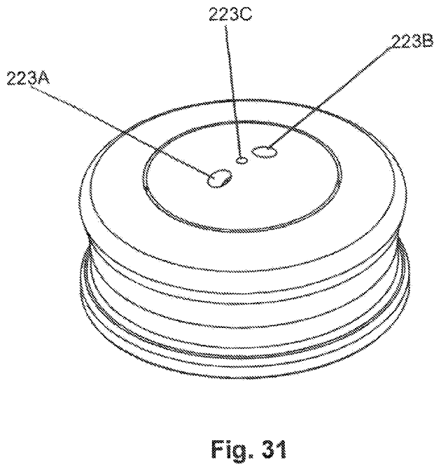

FIG. 31 shows an example perspective view of a second plunger 41 having three outlet openings 223A, 223B and 223C. The three outlets may be arranged in a row. The middle outlet may be smaller than the peripheral outlets. FIG. 32 shows an example cross-section of a second plunger 14 having three outlet openings 223A, 223B, 223C. Like reference numerals illustrate like parts as in FIGS. 27 to 31.

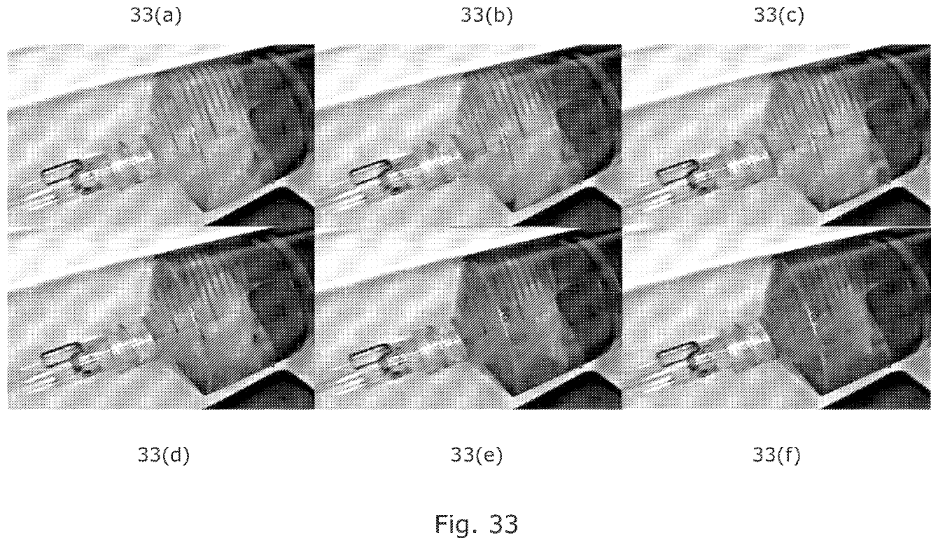

An example of the jets and mixing are illustrated in FIGS. 33A to 33F.

FIGS. 33A to 33F show a priming process, but similar jets and mixing will occur during an infusion. FIG. 33A shows a first time, for instance 0.26 seconds after commencing priming, at which one central and two lateral jets are generated. FIG. 30B shows a second time, for instance after 0.60 seconds, where the central jet has reached the dilution chamber outlet and each of the two lateral jets has reached a mid-sidewall of the dilution chamber. FIG. 30C shows a third time, for instance after 0.8 seconds, in which the central jet has entered a conduit attached to the dilution chamber opening and the two lateral jets have reached respective corners of the dilution chamber. FIG. 30D shows a fourth time, for instance after 1.67 seconds, at which the lateral jets has reached the dilution chamber outlet. FIG. 30E shows a fifth time, for example after 2.6 seconds, at which retrograde mixing of the lateral jets starts to occur. FIG. 30F shows a sixth time, for example after 3.2 seconds, at which retrograde movement of the lateral jets collides with forward movement of the central jet causing further turbulence and mixing.

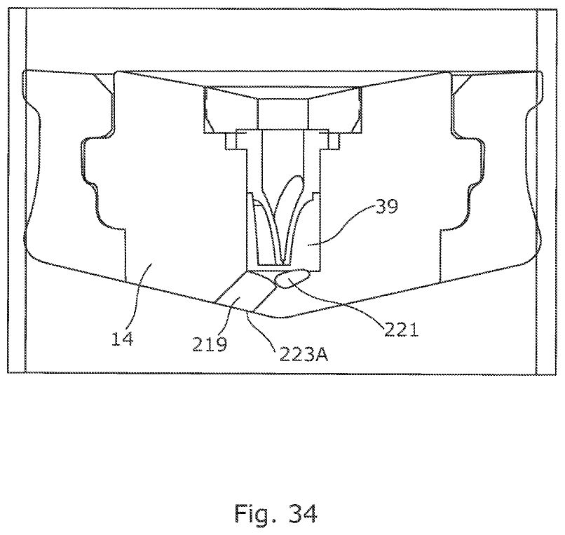

FIG. 34 is a cross sectional diagram of an example of a second plunger 14 having a main body containing a valve 29 inside the main body and wherein the main body has a plurality (e.g. three) of outlet openings and plurality of internal channels, each internal channel leading to a respective outlet opening. Each channel may extend in a different direction or angle. Therefore, although there are three outlets and three outlet channels, as they extend in different directions, in the cross-section of FIG. 34 only the first channel 219 and first outlet 223A and part of the second outlet channel 221 can be seen.

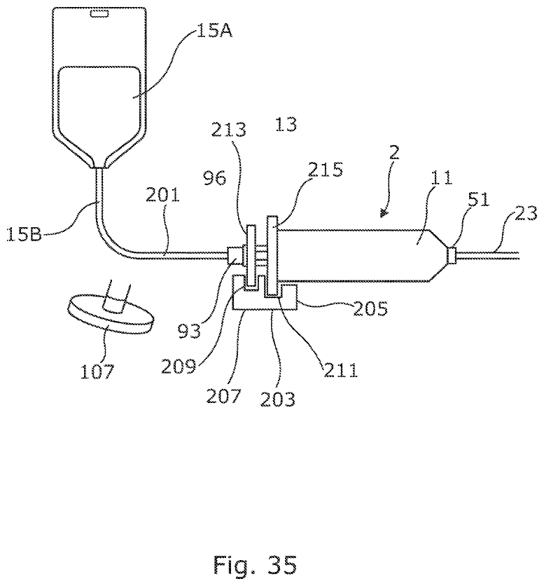

FIG. 35 illustrates a medication delivery system 1, according to some embodiments. The medication delivery system 1 comprises a remote active agent 15A. The remote active agent chamber 15A is in addition to the active agent chamber 15 of the medication delivery apparatus 2. That is, the remote active agent chamber 15A is disposed away from the medication delivery apparatus 2 and may be fluidly connected to the plunger lumen 93 of the medication delivery apparatus 2 described herein by an active agent chamber conduit 201. The active agent chamber conduit 201 may be configured to connect to an outlet of the remote active agent chamber 15A and to the first plunger connector 96.

An external pump 15A may be used to pump pharmaceutical preparation from the remote active agent chamber 15A into the active agent chamber 15 of the medication delivery apparatus 2. In this arrangement, the active agent chamber 15 of the medication delivery apparatus 2 is maintained at a fixed volume. Flow of the pharmaceutical preparation is driven by the external pump 15B, which may for instance be peristaltic pump or other pump. The pump 15B may drive flow of pharmaceutical preparation into the active agent chamber 15 and through the valve 39 (not shown) in the dilution chamber 25 (not shown) and out through the dilution chamber opening 51.

In order to maintain the active agent chamber 15 at a fixed volume, the medication delivery system 1 may comprise a plunger lock 203. The plunger lock 203 fixes the first plunger 13 at a particular location. This enables the pharmaceutical preparation to be delivered into the dilution chamber 25, via the valve 39 for mixing with the diluent and to flow out of the dilution chamber opening for delivery to the patient. As the first plunger does not move, the second plunger will also remain in place and so the volume of the dilution chamber is fixed also.

The plunger lock 203 comprises a body 205. The body 205 comprises a lower surface 207 for resting on a support surface. The body 205 comprises a first groove 209. The body comprises a second groove 211. The first groove 209 is configured to receive a first plunger flange 213 of the first plunger 13. The second groove 211 is configured to receive a container flange 215 of the container 11. The plunger lock 203 is configured to fix the first plunger 13 at a particular location, so that the first plunger 13 is stationary within the container 11.

The external pump may be controlled to drive the pharmaceutical preparation in accordance with a particular dose profile. For instance, the pharmaceutical preparation may be caused to flow at a varying flow rate determined by a function suitable for delivering the desired dose profile when the dilution chamber is of fixed volume. For example, the rate of active agent administration may be governed by the Sadleir Function as described in International Patent Application Number No. PCT/AU2020/051363, the content of which is incorporated by reference in its entirety. In some embodiments, the rate of active agent administration may be governed by the Increased Volume Sadleir Function as described in International Patent Application Number No. PCT/AU2020/051363.

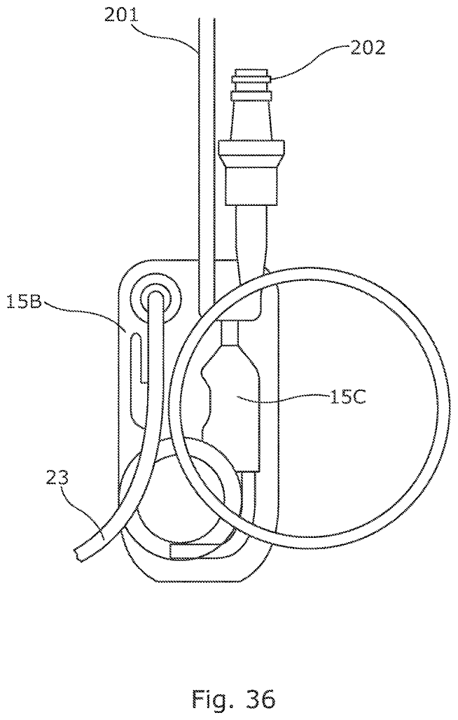

FIG. 36 shows another example in which there may be two separate pharmaceutical preparations administered to a patient. In this case, a primary pharmaceutical preparation may enter a pump 15B through a first inlet 201. The pump 15B has a second inlet which may receive a second pharmaceutical preparation output by a medication delivery device 2 as described in the various examples above. The pump 15B may combine the first and second pharmaceutical preparations and output them through an outlet conduit 23 to a patient (for instance by intravenous administration). The pump 15B may include an air trap 15C for trapping any bubbles formed in the pharmaceutical preparations.

As the dose profile may be sensitive to the initial part of the infusion, in which the dose rate should be kept low, the medication delivery apparatus 2 may be attached to the outlet 202 by a conduit apparatus 175 as described below.

The conduit apparatus allows a priming process which uses a high infusion rate to fill a conduit 23 with diluted pharmaceutical preparation at a desired concentration profile, so that the dose rate can be accurately achieved in the first part of the infusion as the first part of the infusion consists of the diluted pharmaceutical preparation which has been prepared in the conduit 23 as part of the priming process. Without this approach it can be difficult to achieve the desired low, but increasing, dose rate in the first part of the infusion.

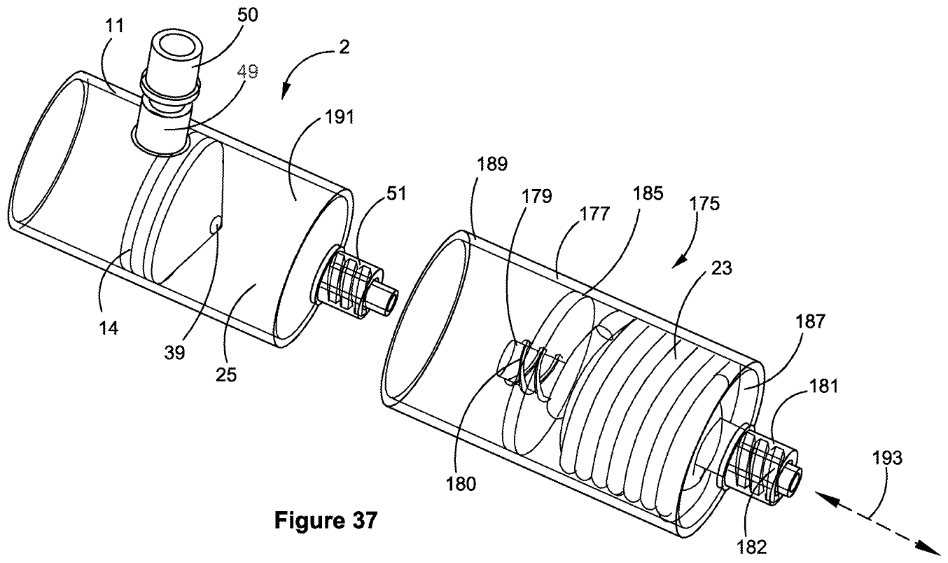

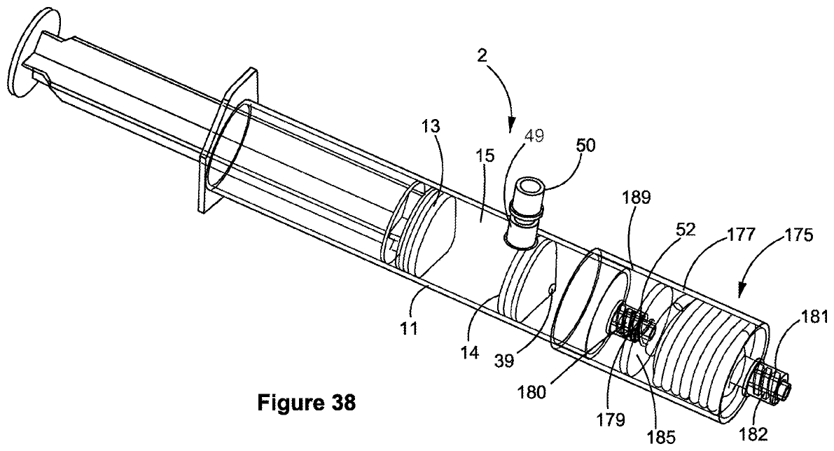

FIG. 37 illustrates a perspective view of the conduit apparatus 175 and a portion of the medication delivery apparatus 2, according to some embodiments. The housing 177 and the conduit 11 are both partially transparent in FIG. 37. FIG. 38 illustrates a perspective view of the conduit apparatus 175 and the medication delivery apparatus 2, according to some embodiments. The housing 177 and the conduit 11 are both partially transparent in FIG. 38.

The housing comprises a first housing port 179. The first housing port 179 comprises a first housing opening 180. The first housing opening 180 extends through the first housing port 179. The first housing port 179 comprises a first connector. The first connector may be a first housing Luer-lock connector. The first housing Luer-lock connector may be a male Luer-lock connector. Alternatively, the first hosing Luer-lock connector may be a female Luer-lock connector. The first housing port 179 is configured to connect to the dilution chamber port 51 of the medication delivery apparatus 2.

The housing 177 comprises a second housing port 181. The second housing port 181 comprises a second housing opening 182. The second housing opening 182 extends through the second housing port 181. The second housing port 181 comprises a second housing connector. The second housing connector may be a second housing Luer-lock connector. The second housing connector may be a second housing Luer-lock connector. The second housing Luer-lock connector may be a female Luer-lock connector. Alternatively, the second hosing Luer-lock connector may be a male Luer-lock connector. The second housing connector is configured to connect to tubing.

The conduit 23 connects the first housing port 179 and the second housing port 181. Specifically, the conduit 23 fluidly connects the first housing opening 180 and the second housing opening 182. The conduit 23 is coiled within the housing 175. Specifically, the housing 175 comprises a conduit chamber 183. The conduit 23 is coiled within the conduit chamber 183. The conduit chamber 183 extends between an intermediate wall 185 and a distal end 187 of the housing 175. The conduit 23 is of a predetermined volume, as described herein.

The housing 175 comprises a collar 186. The collar 186 extends away from the intermediate wall 185, away from the second housing port 181. In particular, the collar 186 extends away from the second housing port 181 in a direction that is generally parallel to a longitudinal axis 193 of the conduit apparatus 175. The longitudinal axis 193 of the conduit apparatus 175 may be generally parallel to the longitudinal axis 21 of the medication delivery apparatus 2, when the conduit apparatus 175 is connected to the medication delivery apparatus 2.

The medication delivery system 1 may comprise any medication delivery apparatus 2 described herein, and the conduit apparatus 175. The collar 186 is configured to engage with the container 11. In particular, the collar 186 may be configured to engage with the container 11 via an interference fit. The interference fit may be between an inner collar surface 189 and an outer container surface 191.

The conduit 23 and/or the conduit apparatus 175 provide a number of significant advantages. When the medication delivery apparatus 2 is initially engaged with the infusion device 3, there is an amount of ‘slack’ in the system. This is because, for example, there may be a lack of compression between an infusion driver of the infusion device 3 (e.g. the infusion device's 3 actuator) and the medication delivery apparatus, compressibility of fluid and components of the medication delivery apparatus 2, or other reasons. The conduit 23 being a predetermined volume (e.g. a predetermined minimum volume), as described herein, can mitigate this ‘slack’.

The medication delivery apparatus 2 can be engaged with (i.e. connected to) the infusion device 3 and the infusion device 3 can be operated to drive fluid from the medication delivery apparatus 2 to an end of the conduit 23. The fluid can then be stopped. As the pharmaceutical composition and/or pharmaceutical preparation is not entering the patient during this period, the flow rate of the fluid is not relevant. Thus, any variation in the flowrate from a predetermined target flow rate is not relevant. Furthermore, completing this process removes the ‘slack’ from the system, which is therefore not an issue once the infusion is started in accordance with the desired flow rate.

Furthermore, the pharmaceutical preparation may not always perfectly mix with the diluent when passing through the valve 39. This may particularly be the case where the flow rate is low, due to the low kinetic energy of fluid passing through the valve 39 (e.g. at the initial stages of an infusion). Thus, the rate at which the pharmaceutical preparation is provided to the patient at the early stages of an infusion are affected. Providing the conduit 23 that is of a predetermined volume (e.g. a predetermined minimum volume) as described mitigates this.

This is because the volume of the mixed (i.e. diluted) pharmaceutical preparation delivered during initial stages of an infusion is low and can be less than the total volume of the conduit 23. As the conduit 23 is primed before it is connected to the patient, the flow rate during this period can be arbitrarily high. Therefore, a clinician can use a flow rate that will improve the mixing of the pharmaceutical preparation with the diluent during priming. The fluid generated during priming will then be stored along the length of the conduit 23. A diluted pharmaceutical preparation will be present at a patient end of the conduit 23, and a concentration of the pharmaceutical preparation will increase closer to the dilution chamber 25.

When the conduit 23 is connected to the patient and a predetermined flow rate program initiated, diluted pharmaceutical preparation generated during the priming step is delivered to the patient. Diluted pharmaceutical preparation that was mixed during an early period of the infusion will be delivered to the patient at a much later stage of the infusion, when the flow rate is higher, and so will have a much lower impact on the delivery rate of the pharmaceutical preparation.

In most cases, the conduit apparatus 175 and double inlet pump of FIG. 36 will not be used. In many cases the medication delivery apparatus 2 is used as the only source of pharmaceutical preparation and the dilution chamber outlet may be connected to a conduit 23 of known volume (e.g. extension tubing) so that priming of the conduit 23 can be done in the same manner as described above before the conduit 23 is attached to the patient to commence the infusion.

It will be understood that while fluid delivery system 1 has been described in the context of a medication delivery system 1, in some embodiments, the fluid delivery system 1 may be used for purposes other than the delivery of a pharmaceutical preparation. For example, in some embodiments, the fluid delivery system 1 is configured to deliver a mixed fluid as part of an industrial process or another process. In these cases, the first fluid that is stored in the first chamber 15 may be a first industrial fluid and the second fluid that is stored in the second chamber 25 may be a second industrial fluid. The fluid delivery apparatus 2 may be configured to controllably deliver a mixture of the first fluid and the second fluid as part of an industrial process or another process. It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

For example, the present disclosure describes a number of embodiments of a medication delivery system 1 and a medication delivery apparatus 2. It will be understood that a particular one of the embodiments of the medication delivery apparatus 2 described herein can include one or more features and/or components of another embodiment of the medication delivery apparatus 2 described herein, without departing from the scope of the disclosure.

1. A medication delivery apparatus comprising:
a first plunger;
a second plunger; and
a container configured to receive the second plunger and at least a portion of the first plunger;
wherein:
the container and the second plunger define a dilution chamber that is configured to receive a diluent;
the container defines a dilution chamber opening;
the first plunger, the container and the second plunger define an active agent chamber that is configured to receive a pharmaceutical preparation;
the second plunger comprises a one-way valve configured to control a flow of pharmaceutical preparation from the active agent chamber to the dilution chamber;
the one-way valve is configured to move from a closed position to an open position upon application of a force exceeding a valve threshold force to an inlet side of the valve; and
the valve threshold force is less than a sum of a break loose force of the second plunger and a break loose force of the first plunger;
wherein the medication delivery apparatus is operable to force pharmaceutical preparation from the active agent chamber through the one-way valve into the dilution chamber to mix with the diluent in the dilution chamber and force the mixed diluent and pharmaceutical preparation out of the dilution chamber through the dilution chamber opening.

2. The medication delivery apparatus of claim 1, wherein the valve threshold force is less than the break loose force of the second plunger.

3. The medication delivery apparatus of claim 1, wherein the first plunger has a concave surface facing the second plunger.

4. The medication delivery apparatus of claim 3 wherein the second plunger has a convex surface facing the first plunger and a convex surface facing the dilution chamber opening.

5. The medication delivery apparatus of claim 1, wherein the first plunger and second plunger are shaped such that when the first plunger is moved into contact with the second plunger there is an air a gap between the first plunger and the second plunger.

6. The medication delivery apparatus of claim 1, wherein a distal end of the container defines the dilution chamber opening, the second plunger is movable towards the distal end of the container up to an end position at which the second plunger cannot be moved further towards the distal end of the container and wherein the second plunger is shaped such that in the end position there is a gap between at least part of the second plunger and the distal end of the container so as to prevent a suction force attaching the second plunger to the distal end of the container.

7. (canceled)

8. (canceled)

9. The medication delivery apparatus of claim 1, wherein the one-way valve is contained inside a main body of the second plunger, the main body includes at least one outlet opening and the main body includes at least one internal channel leading from an outlet of the one-way valve to the at least one outlet opening.

10. The medication delivery apparatus of claim 9, wherein the main body of the second plunger comprises at least two outlet openings.

11. The medication delivery apparatus of claim 10, wherein the at least two outlet openings are configured to generate a first jet of pharmaceutical preparation directed toward a first corner of the dilution chamber and a second jet of pharmaceutical preparation directed toward a second corner of the dilution chamber when pharmaceutical preparation is forced from the active agent chamber through the one-way valve.

12. The medication delivery apparatus of claim 11 wherein the apparatus is configured such that the first and second jets of pharmaceutical preparation rebound from an internal surface of the dilution chamber thereby promoting retrograde mixing of the pharmaceutical preparation with diluent in the dilution chamber.

13. (canceled)

14. The medication delivery apparatus of claim 9, wherein the main body of the second plunger comprises three outlet openings and a middle opening of the three outlet openings is configured to generate a third jet of pharmaceutical preparation directed toward the dilution chamber opening.

15. (canceled)

16. The medication delivery apparatus of claim 1, wherein the second plunger has a length along a longitudinal axis of the container which is at least 9 mm.

17. The medication delivery apparatus of claim 1 in combination with a conduit apparatus, the conduit apparatus comprising:
a conduit apparatus housing including:
a first housing port; and
a second housing port; and
a conduit connecting the first housing port and the second housing port;
wherein:
the first housing port comprises a first connector configured for connection to the dilution chamber outlet opening; and
the second housing port comprises a second connector configured for connection to tubing that is to connect to a patient.

18. The medication delivery apparatus of claim 1, wherein the first plunger comprises a plunger lumen, extending between a first plunger lumen opening and a second plunger lumen opening, for delivering pharmaceutical preparation through the plunger lumen and into the active agent chamber.

19. (canceled)

20. (canceled)

21. A method of preparing the medication delivery apparatus of claim 1, the method comprising:
a) filling the dilution chamber with a diluent; and
b) filling the active agent chamber with pharmaceutical preparation;

22. (canceled)

23. The method of claim 21 wherein the active agent chamber is filled with active agent prior to the dilution chamber being filled with diluent.

24. The method of claim 21 wherein the active agent chamber is filled with active agent via a plunger lumen of the first plunger.

25. (canceled)

26. A method of preparing the medication delivery apparatus comprising an active agent chamber, a dilution chamber, a one-way valve connecting the active agent chamber and the dilution chamber and a dilution chamber opening, the method, comprising:
c) connecting the medication delivery apparatus to an infusion driver;
d) attaching extension tubing of a known predetermined volume to the dilution chamber opening of the medication delivery apparatus; and
e) priming the medication delivery apparatus by causing pharmaceutical preparation to pass from the active agent chamber through the one-way valve into the dilution chamber, mix with diluent in the dilution chamber and then exit through the dilution chamber opening into the extension tubing, such that the extension tubing of known predetermined volume is filled with diluted pharmaceutical preparation which is to form a first part of the infusion and wherein a concentration profile of the diluted pharmaceutical preparation in the extension tubing is in accordance with a desired dose profile for a first part of the infusion.

27. (canceled)

28. (canceled)

29. A medication delivery system comprising:
the medication delivery apparatus of claim 1; and
an infusion device;
wherein the infusion device comprises:
at least one infusion device processor; and
infusion device memory storing program instructions accessible by the at least one infusion device processor, and configured to cause the at least one infusion device processor to control the medication delivery apparatus to deliver the pharmaceutical preparation to a patient in accordance with a predetermined dose profile.

30. (canceled)

31. The medication delivery system according to claim 29 wherein the system further comprises an extension tubing of a known predetermined volume to the medication delivery apparatus and the processor is configured to perform a priming process prior to a start of the infusion, wherein the priming process comprises:
priming the medication delivery apparatus by causing pharmaceutical preparation to pass from the active agent chamber through the one-way valve into the dilution chamber, mix with diluent in the dilution chamber and then exit through the dilution chamber opening into the extension tubing, such that the extension tubing of known predetermined volume is filled with diluted pharmaceutical preparation which is to form a first part of the infusion and wherein a concentration profile of the diluted pharmaceutical preparation in the extension tubing is in accordance with a desired dose profile for a first part of the infusion.
